(19) United States
(12) Patent Application Publication
(10) Pub. No.: US 2003/0023385 A1
(43) Pub. Date: Jan. 30, 2003

(54) Statistical analysis method for classifying objects

(76) Inventor: Emmanuel Lazaridis, Tampa, FL (US)

Correspondence Address: Jeff Lloyd,, Esq., Saliwanchik, Lloyd & Saliwanchik, 2421 N.W. 41st Street, Suite A-1, Chicago, IL, 32606-6669, US

(21) Appl. No.: 09/913,498

(22) PCT Filed: Feb. 5, 2001
(86) PCT No.: PCT/US01/03616

(51) Int. Cl. G01N 33/48; C12Q 1/68; A01N 43/04

(52) U.S. Cl. 702/19; 435/6; 514/44

(57) ABSTRACT

An information computational method for classifying multivariate datasets to identify latent (unobservable) properties of members of a sample, which properties are then used for classification. The method comprises a novel combination of statistical and fuzzy logic methods whereby the latent classes of each object are identified according to the formula:

ƒ( [symbol] ⱼ_{₁}_{, . . . , j}_{_{K}})|{(jₖεSₖₘ_{ⱼₖ}}ₖ₌₁^{K}˜G[h(k,jₖ,{{Sₖₘ}ₘ₌₁^{M}^{ₖ}}ₖ₌₁^{K})]

wherein kε{1, . . . , K} indexes the directions of the multidimensional space; jₖε{1, . . . , Nₖ} identifies an object in direction k; Nₖ is the number of objects in principal direction k; [symbol] ⱼ_{₁}_{, . . . , j}_{_{K}} is a vector of one or more observations on a set of objects {j₁, . . . , j_{K}}; mε{1, . . . , Mₖ} indexes latent classes in direction k with Mₖ being the number of latent classes in direction k; Sₖₘ is a latent class m in direction k; G[·] is a specified univariate or multivariate distribution; f(·) and g(·) are specified functions; and the method calculates the likelihood that each object of interest belongs to each identified latent class. The invention addresses a variety of informatics problems, particularly in the field of biology, and permits a user to make reasonable inferences about underlying cause-effect relationships, such as the underlying biology of gene-expression patterns.

### 1. FIELD OF THE INVENTION

The present invention is an information computational method for defining cause-effect linkage. More specifically, the present invention relates to a process for analyzing multivariate sets of data by way of a marriage of technology arising from studies of fuzzy logic and statistics. Thus, this invention addresses a variety of informatics problems, particularly as relating to the field of biology. For example, this invention improves our ability to comprehend data from experiments employing a vast number of genes by algorithmically clustering similar data points. The method identifies latent (unobservable) properties of members of a sample, which properties are then used for classification.

### 2. BACKGROUND OF TH INVENTION

The traditional method for predicting the clinical characteristics of a cancer comprises providing a tissue sample from a subject; recording predictive parameters, specifically one or more morphometric descriptors or the results of a specific staining assay; and predicting the metastasizing potential of the cancer by a statistical comparison of the recorded predictive parameters with corresponding parameters of a reference sample. Despite advances in understanding of genetic events that underlie the development of cancers, the capacity to predict metastatic potential is limited, and the mechanisms underlying the process are poorly understood. At the present time, the traditional method in particular does not predict metastasis potential with sufficient certainty. Thus, the capacity to predict metastasis through analysis of gene expression patterns would be an important advance, providing great insight into the mechanisms responsible for this complex process and identifying new targets for therapeutic intervention.

Precise dissection of gene expression under a particular external influence or point in time can be achieved in high-throughput fashion by collecting data using cDNA microarray-technology. Microarrays are microscope slides, membranes, or chemically-modified silicon surfaces containing hundreds to tens of thousands of immobilized DNA samples. This array of cDNA-spots can be probed with fluorescently labeled cDNAs, which are obtained by RT PCR from total RNA pools corresponding to the test and reference biological sources. Following a hybridization step with two dye-tagged probes corresponding to reference and test cDNAs, the microarray is scanned to generate two images, each one corresponding to one of the dye “colors”. Thus, the level of intensity at each particular point in each image corresponds to the amount of probe, tagged with the corresponding color dye, at that position. These images are typically captured as 16-bit TIFF-formatted files containing as many as 20 million-picture elements (pixels), which must be analyzed statistically to reveal patterns and correlations among the hybridization of the many gene probes present.

All current methods of image-processing create a bottleneck of information. Typically, the image pixels are first converted to quantified gene expression data. The data obtained from the images have to be further processed and converted into biologically-meaningful information, in order for one to draw conclusions about biological processes occurring at the molecular level. Array image processing is often employed to measure the intensity of the array spots and to quantify their expression levels. The resulting data therefore consist of an array of numbers, each of which relates to a specific spots and its specific gene.

In practice, the process of spot identification is carried out either with human eyes and judgement or by computer-vision programs. The first approach is expensive, error-prone, exhaustive and prohibitive for large-scale experiments. Thus it is clear that a sophisticated machine-vision and analysis program is necessary to overcome these and other information bottle-neck problems. After the positioning of the array and identification of the object of interest, location and size, such a program processes the image. An image-segmentation algorithm is typically used to appropriately identify and segregate the pixels associated with each spot signal area from its local background and possible contaminants. Regardless of what algorithm is used to identify and quantify the image intensities related to gene expression, the resulting data must be modeled if one is to understand what biology underlies variation in gene expression over multiple microarray measurements. Such gene expression patterns can be extremely complex. For example, existing knowledge has implicated numerous classes of proteins and associated processes (angiogenesis, adhesion, invasion, growth) in the development of tumor metastases. Clustering methods (a method frequently used to analyse and classify multivariate datatsets), by themselves, are not adequate in resolving molecular fingerprints linked to colon cancer metastasis. Several research groups have come to this conclusion. For example, Tibshirani, et al., developed a new technique based on principal components analysis, which they termed gene shaving, to enhance the results of hierarchical clustering (see Tibshirani, R., et al., Clustering methods for the analysis of DNA microarray data. Preprint, International S-Plus User Conference, 1999: pp. 1-23). Their goal was to discover a small set of genes whose expression across tissue samples best described the directions of greatest observed variability over all the genes in the data set, without formally quantifying the grouped expression patterns themselves.

Fuzzy logic was proposed to be a superset of conventional (Boolean) logic that has been extended to handle the concept of partial truth—truth values between “completely true” and “completely false”. It was introduced by Dr. Lotfi Zadeh of UC/Berkeley in the 1960's as a means to model the uncertainty of natural language. Fuzzy logic is implemented most commonly in control system design. Fuzzy logic-based systems are found in a rapidly growing number of consumer appliances (from dishwashers to video cameras), as well as in automobile engines and transmissions and industrial equipment. The intuitive nature of the fuzzy-based system design saves engineers time and reduces costs by shortening product development cycles and making system maintenance and adjustments easier. The use of fuzzy logic for creating decision-support and expert systems in particular has grown in popularity among management and financial decision-modeling experts. Others are applying fuzzy logic to problems of pattern recognition, economics, data analysis, and other areas that involve a high level of uncertainty, complexity, or nonlinearity. For example, fuzzy logic decision tree algorithms are widely used to perform handwritten character recognition in palm-held computers.

Semantically, the distinction between fuzzy logic and probability theory has to do with the difference between the notions of probability and degree of membership. Probability statements are statements about the likelihoods of outcomes: in this framework, an event either occurs or does not. But with fuzziness, one cannot say unequivocally whether an event occurred or not, and instead one tries to model the extent to which an event occurred. Although disagreements between statisticians and fuzzy-set theorists as to the consequences of this distinction have not been resolved to everyone's satisfaction, the developer of the present invention and his colleagues have demonstrated that fuzzy logic structures can be employed by statisticians when degrees of membership are treated as probabilities. In so doing it has been shown that the resulting mathematical forms are poor ones to use in addressing most multidimensional applied problems. Thus, fuzzy logic methods alone do not provide efficient analytical tools for large, multivariate data sets.

Available statistical models for the analysis of multidimensional data also have substantial drawbacks. Typically, statisticians employ multivariate probability distributions to model a multidimensional space. Such distributions are often difficult to employ or inadequate to reflect the underlying structures in the data. Alternatively, classification methods based on splines or classification trees are employed. Although they are easy to use, these also perform poorly in reflecting structure in commonly found non-linear spaces.

The invention described herein below overcomes the problems inherent to fuzzy logic and traditional statistical methods, by combining elements of both to create more tractable model forms. This novel method is shown to be a significant improvement over standard methods for the analysis of many kinds of data, including the analysis of microarray data as described above. The present invention augments substantially, and is superior to, existing methodologies in current use, such as hierarchical clustering and principal components analyses, to analyze these kinds of data. Thus, the present invention provides solutions to the specific problems described above related to the analysis of large, multivariate datasets. These, and other advantages, will become apparent to one of skill in the art upon reading the following disclosure and examples.

### 3. SUMMARY OF THE INVENTION

The present invention provides a novel method for classifying multivariate datasets and for making reasonable inferences about the underlying cause-effect relationships, for example the underlying biology of gene-expression patterns. Whereas other available methods perform only one-dimensional classification, the novel approach classifies these data along two or more dimensions. Generally the novel method is designed to address multi-dimensional classification problems, where two or more dimensions are available for classification. This approach rests on a novel combination of statistical and fuzzy logic methods.

This invention addresses the general problem of making sense of huge datasets and making sensible connections between cause-effect events that may only be indirectly observable. A specific example arises in the context of the problem of predicting cancer metastatic potential through the molecular analysis of human tumors with the goal of determining whether patterns of gene expression that portend metastasis may be deciphered from tumor specimens. Thus, the capacity to predict metastasis through analysis of gene expression patterns is an important advance, providing great insight into the mechanisms responsible for this complex process and identifying new targets for therapeutic intervention. Data from human colon cancer metastatic variant cell lines serves as a model used to help decode complex patterns of gene expression. When these data are supplemented by information from patient colon cancer and normal tissues, patterns of gene expression linked to metastasis may be deciphered. A successful deciphering method identifies in a genomic library a gene or set of genes linked to metastatic properties of a cancer. It also works in the clinic to classify patients into those with high versus low metastatic potential. In one embodiment, the present invention addresses the metastasis problem. Because gene expression can be induced or repressed, the method is designed to assess patterns of gene expression that include both over- and under-expressed genes.

The present invention employs multidimensional, generalized, latent class structures in a statistical framework to identify and describe patterns. Examples of data amenable to analysis by this method can be found in studies of gene expression (genes in the first dimension and tissue samples in the second, where the data measure RNA or protein expression). In a manner analogous to an extension of two-way ANOVA, statistically-significant interactions between the dimensions are identified. In the context of gene expression studies, these indicate pathways that are up- or down-regulated differently among different cells, tissues or experimental conditions. The present invention builds models for appropriate sets of data along a nested hierarchy of complexity. In the context of gene expression studies, the method initially assumes that the underlying biological pathways may be on or off by degrees, and that each gene's expression may be controlled by one or more pathways in a stochastic mixture. Based on residual analyses and other model diagnostic procedures (seeking ways in which a chosen model does not fit the data), or on external scientific information, initial models are enhanced by adding pathway interactions and physical mixing parameters, allowing one to evaluate contributions of non-stochastic mixing. For example, recent studies have demonstrated that whereas MYC-MAX complexes activate transcription, MAD-MAX complexes repress transcription (see incorporated by reference U.S. Pat. No. 5,811,298 for a general disclosure of MYC-MAX and MAD-MAX). The expression of transcription factors depends on the quantities of each of these complexes and their components. This kind of situation is not well represented by a model that assumes independence among the complexes which activate or repress transcription. The present invention includes a feedback loop for building more complicated models to address such issues. In addition, correlational information, such as pathology data concerning the observed mixing of cell types in a tissue sample, can be incorporated into the model. By establishing the effects of cellular contamination on the collected gene expression data and o adjusting for these in a manner similar to ANCOVA prior to estimation of tumor-type differences, unwanted inter-person expression variability is substantially reduced. Examples that illustrate the ability of the present invention to decipher patterns of gene expression across microarrays, and to extract molecular fingerprints associated with particular tissue types, are given hereinafter.

In contrast to known methods, the present invention seeks to explicitly model patterns, across genes, tissue samples, or time, and to determine the probability that each gene or sample is a member of each of the set of estimated latent gene or sample classes, respectively.

The present invention is not limited to colon cancer and is useful to construct models for screening a wide variety of neoplastic diseases, including both solid tumors and hemopoietic cancers. Exemplary neoplastic diseases include carcinomas, such as adenocarcinomas and melanomas; mesodermal tumors, such as neuroblastomas and retinoblastomas; sarcomas, such as osteosarcomas, Ewing's sarcoma, and various leukemias and lymphomas. Of particular interest are adenocarcinomas of the breast, ovaries, colon, stomach, liver and lung. Depending on the neoplastic disease, an appropriate patient sample is obtained. In the case of solid tumors, a tissue sample from the surgically removed tumor is obtained and prepared for testing by conventional techniques. In the case of lymphomas and leukemias, leukemic cells of blood or bone marrow or lymphoid tissues are obtained and appropriately prepared. Other patient or host samples, including urine, serum, sputum, cell extracts, etc. are also useful under certain conditions. As defined herein the term host denotes a mammal, preferably a human who may have a disease or be suspected of having a disease. Accordingly the present invention is used to identify genes linked to the disease via analysis of cell or tissue samples collected from a multiplicity of hosts, and to identify disease status in individuals suspected of having the disease. The preferred steps in this method include organizing one or more measurements on each of the cell or tissue samples, or over a series of experimental or observational conditions, in an array; allowing genes to form a first dimension in a multidimensional space; allowing cell or tissue samples to form a second dimension in a multidimensional space; identifying latent classes of genes in the first dimension and latent classes of cell or tissue samples in the second dimension; calculating the likelihood that each gene is a member of each latent class identified for the first dimension; and calculating a likelihood that each cell or tissue sample is a member of each latent class for the second dimension.

Another preferred embodiment of this invention entails the determination of correlation between protein expression and RNA expression in deciphering patterns portending to cancer metastasis, and to define associated phosphorylation-signaling events. Representative cancers include but are not limited to leukemias such as acute leukemia; acute lymphocytic leukemia; acute myelocytic leukemia; myeloblastic; promyelocytic; myelomonocytic; monocytic; erythroleukemia; chronic leukemia; chronic myelocytic (granulocytic) leukemia; chronic lymphocytic leukemia; Polycythemia vera; lymphoma; Hodgkin's disease; non-Hodgkin's disease; multiple myeloma; Waldenstrom's macroglobulinemia; heavy chain disease; solid tumors sarcomas and carcinomas including fibrosarcoma; myxosarcoma; liposarcoma; chondrosarcoma; osteogenic sarcoma; chordoma; angiosarcoma; endotheliosarcoma; lymphangiosarcoma; Kaposi's sarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; ependymoma; craniopharyngioma; medulloblastoma; pinealoma; hemangioblastoma; acoustic neuroma; oligodendroglioma; meningioma; melanoma; neuroblastoma; retinoblastoma; and other types of tumors including virally induced cancers.

The present invention also pertains to methods of identifying disabilities, medications, comorbidities, laboratory results, and clinical characteristics linked to processes of aging, disease, cancer, diabetes, pregnancy or other clinical conditions in humans. The preferred steps in this method include describing in a matrix or array one or more measurements of said disabilities, medications, comorbidities, laboratory results, clinical characteristics and so on, on each of a set of human subjects; allowing human subjects observed or treated under differing observational or experimental conditions to form the first dimension in the associated multidimensional space; allowing measurements to form one or more additional dimensions; identifying latent classes of human subjects in the first dimension, and latent classes of measurements in the second direction; and calculating the likelihood that each human subject is a member of each identified latent class for the first direction while also calculating, simultaneously or serially, the likelihood that each measurement is a member of each identified latent class in its associated dimension.

Without limiting to above embodiments other applications are easily recognizable to one skilled in the art. These include but are not limited to studies of cellular processes associated with cell metabolism, cellular damaging agents, biological pathways, protein expression, drug effects, and linkages between one or more other expressed genes. They also include studies of processes of aging, physical processes in inorganic substances, chemical substances with pharmacological activity, and performance of financial vehicles such as stocks. In other words, the embodiment of the invention is the generation and analysis of cause-effect and stimulus-response profiles by a computer-based analytical algorithm.

Another preferred embodiment of the invention is its combination of simultaneous mRNA and proteomic analysis of meticulously selected and acquired human tumor specimens to identify molecular patterns portending metastasis. In this manner information is derived by combining two comprehensive technologies (microarray analysis with proteomic analysis) on identical or disparate cell or tissue specimens. Additional sources of information may also be added. These sources may comprise any of a vast number of measurable parameters including but not limited to morphometric descriptors such as the optical density of a measured object, object size, object shape, object color, amount of DNA or RNA, X-Y-Z coordinates in a multidimensional space, angular second moment, contrast, correlation, difference moment, inverse difference moment, sum average, sum variance, sum entropy, entropy, difference variance, difference entropy, maximal correlation coefficient, coefficient of variation, peak transition probability, diagonal variance, diagonal moment, second diagonal moment, product moment, triangular symmetry, sum entropy, standard deviation, cell classification, e.g., 1=Hypodiploid, 2=Diploid, 3=S-Phase, 5=Tetraploid, and 6=Hyperploid, blobness, perimeter, DNA index, maximum diameter, minimum diameter, elongation, run length, and any number of other measurable parameters known in the art. The preferred steps in this method include organizing one or more measurements on each of the cell or tissue samples, or over a series of experimental or observational conditions, in an array; allowing genes to form a first dimension in a multidimensional space; allowing cell or tissue samples to form a second dimension; allowing methods of expression measurement to form a third dimension; allowing other measurements to form one or more additional dimensions; identifying latent classes of genes in the first dimension, latent classes of cell or tissue samples in the second dimension, and latent classes of measurements in the third and further dimensions as appropriate; calculating the likelihood that each gene is a member of each latent class identified for the first dimension; and calculating a likelihood that each cell or tissue sample is a member of each latent class for the second dimension. In this context, a further embodiment of the present invention contemplates means for identifying the disease state of a subject providing one or more cell or tissue samples. The preferred steps in this method include extracting from these samples experimental data including information about genes or gene expression; and calculating the likelihood that the subject is in each latent class previously identified in an experimental or observational study as above.

The invention also addresses the analysis of human colon cancer metastatic variant cell lines. To date, 19,200 different genes on at least five cell lines (KM12 C, KM12 SM, KM12 L4A, SW 480, and SW 620) have been analyzed, with identification of approximately 100 differentially expressed genes common to all three high-metastatic variants. While specific numbers are clearly not limiting in any way, it is contemplated that the present invention is generally useful for identifying among a plurality of genes of known and unknown function those genes that are linked to a condition of interest by providing a mathematical model which utilizes the input data to set rejection margins; entering experimental data from the plurality of genes of known and unknown function; and selecting to genes linked to the condition of interest based on an acceptability criteria of the mathematical model.

Another embodiment of the invention addresses the effect of warm or cold ischemia and normal cell contamination on microarray or proteomics analyses. For example, the invention is being applied to studies of ischemic effects in human colonic mucosal samples. It is critical to rigorously evaluate the critical effects of in vitro and in vivo ischemia associated with the surgical and pathological procedures used to extirpate the tumor specimens and the contribution of normal infiltrating cells on the analysis of gene expression. Thus, a preferred embodiment of this invention entails the determination of ischemia susceptible genes. In one experiment, data were collected regarding 1420 genes in a 2400 element array, using cells subjected to ischemia lasting from 5 to 60 minutes. Because tissues historically collected and stored in tissues banks at many medical institutions were collected without regard to the length of time in which tissues were subjected to ischemia, it is important that a means be established for adjusting data obtained from these tissues for potential ischemic effects. It is contemplated that the present invention is able to identify genes that are particularly susceptible to ischemia, and that it adjusts associated data for ischemic effects.

Another preferred embodiment provides for re-analysis of published or publically-available data sets derived from various cell or tissue studies. For example, these might include microarray data from time-course monitored serum-stimulated fibroblasts, or from human normal and cancerous colon tissues. The present invention has been applied to analyze 8613 different expressed genes in serum-stimulated fibroblasts, clearly discriminating genes over time with the identification of a refined set of patterns. It has also been applied to the analysis of almost 2000 gene products to not only discriminate between normal and tumor tissues, but in addition, to identify molecular fingerprints of multiple kinds of tumor tissues. Tumors having different molecular expression patterns, arising from different aberrations of the genetic code, manifest different characteristics that are important for cancer control and therapies. It is contemplated that the present invention identifies and distinguishes among important tumor subclasses, by deciphering molecular fingerprints that discriminate among different cell or tissue samples.

### 4. BRIEF DESCRIPTION OF THE FIGURES.

FIG. 1 shows an example hybridization image resulting from use of Cy3 and Cy5 labeled probes. Hybridization of Cy3 and Cy5 labeled probes to a region of a 19,200-element human array from TIGR. A Cy3-labeled (lighter spots, green) probe from the KMI 2C colon tumor cell line and a Cy5-labeled (darker spots, red) probe from the KM12L4a were prepared and competitively hybridized to the array. Genes up-regulated in KM12L4 are false-colored darker (red); those genes down-regulated appear lighter (green).

FIG. 2A shows results of differential gene expression analysis in various colon carcinoma cell lines. Three replicas of a 19,200 element microarray hybridization experiment are analyzed to generate a consensus for the indicated pairs of cell lines. Consensus sets are produced by selection genes showing consistent patterns of differential expression across multiple cell lines.

FIG. 2B displays prg gene expression which is up-regulated by about 8-fold in all analyzed samples.

FIG. 3 shows patterns from analysis, using the present invention, of serum-stimulated fibroblasts as negative and positive gene expression patterns in a time dependent manner. Serum-stimulation patterns derived using the present invention, including median estimates and 95% confidence intervals from analysis of serum-stimulated fibroblasts, demonstrating both time-dependent increases and decreases in gene expression, as represented by positive (mostly left panel, red) and negative expression (mostly right panel, green) patterns.

FIG. 4 displays comparative results from TaqMan and Microarray assays.

FIG. 5 shows adjusted estimates of Mast and B4-2 expression derived by the present 5 invention from the microarray data FIG. 6 illustrates a time course with genes clustered by temporal expression pattern (5, 10, 15, 15, 30, 40 or 60 min of subjecting to warm ischemia). Different RNAs degrade with unique half-lives. As members of the initial RNA population become extinct, the relative levels of RNAs in the sample change. This is reflected in an apparent time-dependent pattern of expression when assayed using cDNA microarrays. mRNA levels from human colon mucosal samples subjected to 5, 10, 15, 30, 40, or 60 minutes of wann ischemia are measured relative to a reference cell line (KM12C) using cDNA microarrays containing 2,400 distinct elements. The data from 1420 elements is shown. Each row corresponds to a specific gene and each column a particular time point measurement. Red (darker) elements are expressed at an elevated level relative to the reference sample, green (lighter) at a depressed level. (a) Expression levels following 5, 10, 15, 30, 40, or 60 minutes of warm ischemia measured relative to a reference cell line (KM12C); in (b), the contrast and brightness have been adjusted to aid in visualization. Data in (c) can be used to infer the expression levels of each gene relative to its level at any other time point. In (c) data from times 5-15 min, Al, are averaged and displayed next to averaged data from 20-60 min, A2. In (d), the ratio of the averaged data from 5-15 min (A1)/the averaged data from 20-60 min(A2) is displayed. Representations in (c) and (d) demonstrate that differences in gene expression do occur over the course of 60 min while EtBr-stained gel analyses and Northern analyses for GAPDH show no perceptible change over the same time course for normal mucosa (e) and tumor specimens (f).

FIG. 7 displays hierarchical clustering of time-course data for ischemic decay according to Eisen analysis

FIG. 8 shows 3 types of patterns of time-dependent increases and decreases in gene expression, resulting from analysis using the present invention Ischemia patterns derived using the present invention, including median estimates and 95% confidence intervals from analysis of ischemia data at 6 time points (5 minutes to 1 hour), demonstrating various time-dependent increases and decreases in gene expression, as represented by positive (red, darker) and negative (green, lighter) expression patterns.

FIG. 9 shows the interaction estimates (γₘₗ) between latent gene classes and latent tissue classes.

FIG. 10 shows a representative 2-D gel of proteins after exposure to peroxisome proliferators.

FIG. 11 shows phosphorylated and non-phosphorylated versions of a protein in a 2-D gel.

FIG. 12 shows use of KM12C cell line as a reference standard initially and upon optimization and switching reference.

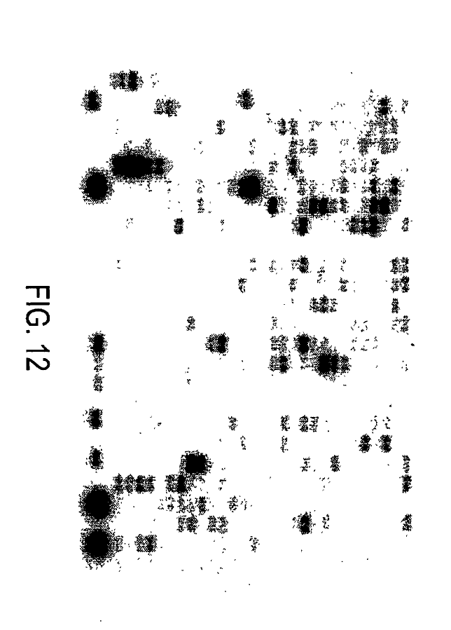

FIG. 13 shows the evolutionary path which allows to predict metastasis on the basis of shared traits.

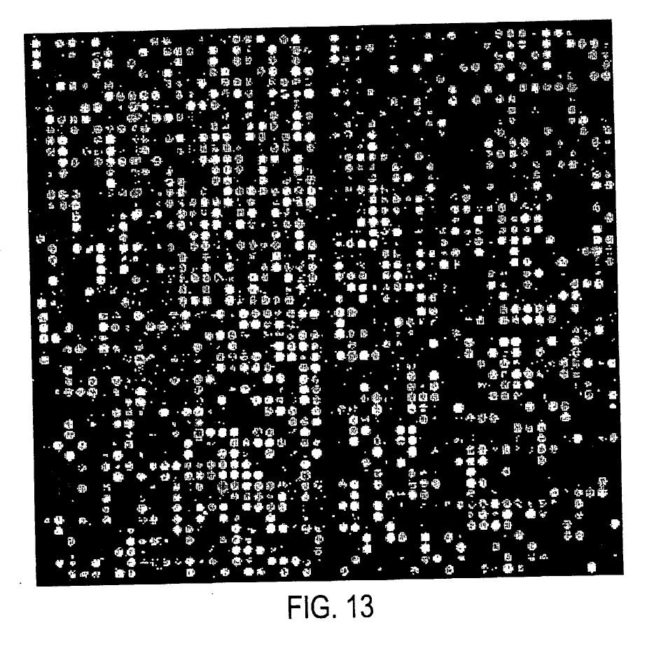

FIG. 14 shows the principle of shared (S) metastasis-predisposing traits as illustrated by the Venn diagram.

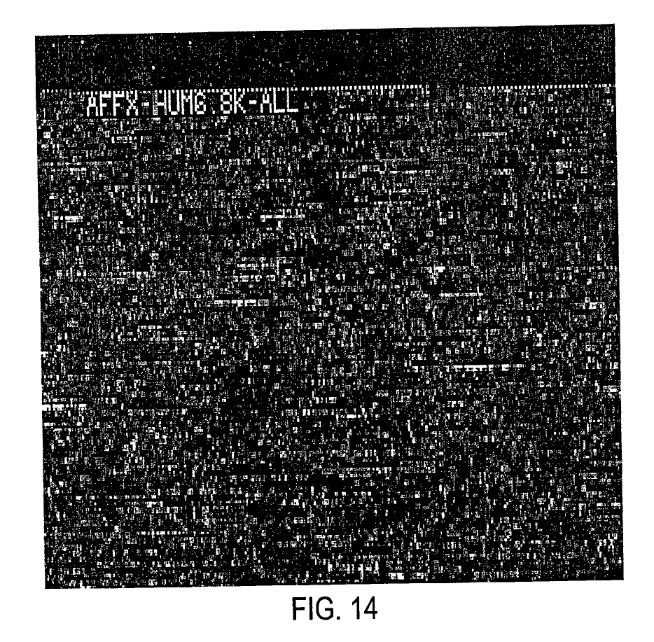

FIG. 15 shows the principle of reduction of the number of gene sets shared by primary (P) metastatic (M) and cancer cell line (CL) variants.

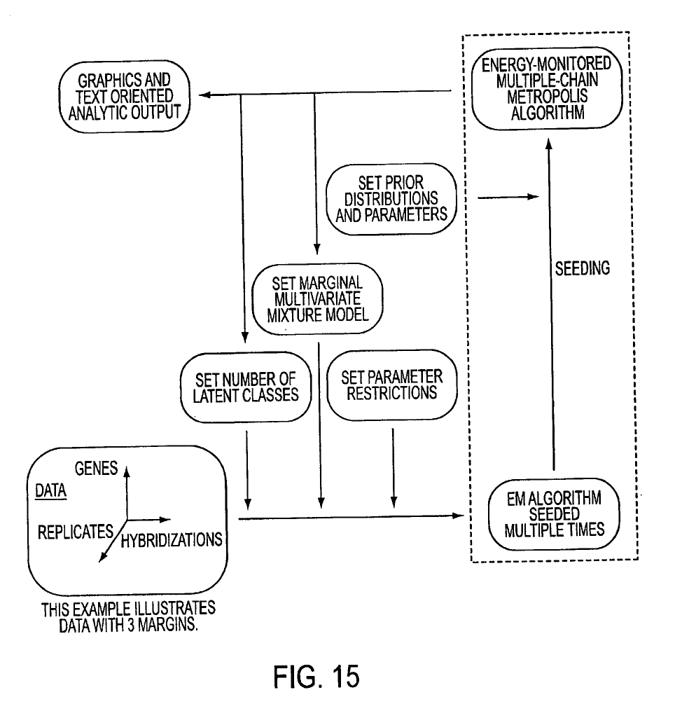

FIG. 16 shows Western analysis of HCT116 cancer cells transfected with the temperature sensitive V138 construct.

FIG. 17 shows portions of gene sets shared between primary (P) and metastatic (M) tumors in tumors harboring P53.

### 5. DETAILED DESCRIPTION OF THE INVENTION

According to the features in the described preferred embodiments, the present invention identifies potentially important patterns in multidimensional data that are not disclosed using standard methods. As a general method for classifying a plurality of objects, either simultaneously or sequentially, the present invention comprises the following steps: one or more observations are collected on one or more sets of objects; the observations are ordered in a matrix or an array representing the multidimensional space for analysis; a model is chosen to represent the multivariate structure of the data, which has a mathematical representation chosen from among models of the form

$$f ( {\vec{Y}}_{{j}_{1} , … , {j}_{K}} ) \vert {\{ {j}_{k} ∈ {S}_{k {m}_{{j}_{k}}} \}}_{k = 1}^{K} \sim G [ h ( k , {j}_{k} , {\{ {\{ {S}_{k m} \}}_{m = 1}^{{M}_{k}} \}}_{k = 1}^{K} ) ] ;$$

latent classes are identified along one or more of the constructed dimensions; the likelihood is calculated that each object of interest belongs to each of the identified latent classes along its specific dimension; objects are assigned among the identified latent classes along each dimension according to the estimated likelihoods. In this formula kε{1, . . . , K} indexes the directions of the multidimensional space; jₖε{1, . . . , Nₖ} identifies an object in direction k; Nₖ is the number of objects in principal direction k; \overline{Y}ⱼ_{₁}_{, . . . , j}_{_{K}} is a vector of one or more observations on a set of objects {j₁, . . . , j_{K}}; mε{1, . . . , Mₖ} indexes latent classes in direction k with Mₖ being the number of latent classes in direction k; Sₖₘ is a latent class m in direction k; G[·] is a specified an univariate or multivariate distribution; and f(·) and g(·) are specified functions.

In the context of microarray experimentation, models in the above family of the form

log(Yᵢⱼ)iεSₘ,jεGₗ˜N[ƒ(t_{il,α}ₘᵢ,βᵢⱼ,γₘₗ),σ²]

have been found useful in studies to identify one or more genes in molecular fingerprints linked to a cellular phenotype, to a biological pathway, to transcriptional effects of a drug, to metastasis potential of human colon cancers, and to differences between multiple types of tumors. In the above formula, N [·] refers to a Gaussian distribution; Sₘ is a latent class m in the first dimension, referring to a category of cell or tissue samples; Gₗ is a latent class I in the second dimension, referring to a category of genes; and ƒ(tᵢₗ,αₘₗ, βₗⱼ,γₘₗ) is a function of parameters related to a sample category (α's), gene category (β's), sample by gene category interactions (γ's), and gene-specific intensity expression intensity (t's ). These models provide the present invention with the capacity to assign probability statistics to the patterns or pathways to which genes are assigned. Thus, the present invention employs two-dimensional, generalized, latent class structures in a statistical framework to identify and describe patterns among genes (first dimension) and microarray hybridizations (second dimension). In a manner analogous to two-way ANOVA, statistically significant interactions between the two dimensions are analyzed and reveal pathways that are up- or down-regulated differently among different microarray experiments. Examples using each of the individual models incorporated in this approach are found in Lazaridis, E. N., Discrimination and Classification Using Conditionally Independent Marginal Mixtures, in Department of Statistics. 1994, University of Chicago: Chicago, Ill. p. 231; Lazaridis, E. N. Should we be fuzzy Bayesians? 1995. Orlando, Fla.: American Statistical Association; Lazaridis, E. N., A Bayesian evaluation of fuzzy logic in a classification problem. Communications in Statistics: Stochastic Models, 1999. 15(3), publications which are incorporated herein by way of reference. The present invention is distinguished from other prior art approaches used to explore microarray data in that it uses flexible, non-parametric probability structures to identify and quantify data patterns. Non-probabilistic algorithms estimate patterns based on relative distances of the expressed genes from one another in the multidimensional array space. Depending on what metric or space-exploring algorithm one chooses, one obtains very different results across methods, with no formal means for deciding among competing models. This is of especial concern in the context of gene discovery, where poor assignment of expressed genes with unknown functions to the wrong patterns may lead to superfluous or misallocated laboratory experimentation. Formal statistical understandings of large gene sets are also necessary if microarray-like technologies are ever to mature beyond the exploratory laboratory research setting. For example, early detection of metastatic potential may ultimately require evaluation of the joint expression of hundreds of genes in order to predict disease status with high sensitivity and specificity. The expression pattern of any particular sample would need to be compared to and adjusted for possibly dozens of identifiable, highly multidimensional expression fingerprints. Formal but flexible probabilistic models are natural and strong candidates for this kind of work.

Thus, the specific advantages of the present invention include the following: (1) it incorporates exploratory models, with intentionally weak structural assumptions so as not to impose artificial patterns on the data, making them very useful tools for complex data exploration; (2) it estimates broad expression patterns over genes and over cell or tissue samples, allowing one to quantitatively determine new biological knowledge; (3) it assigns to individual genes probabilities of membership in specific patterns, allowing one to quantify uncertainty associated with allocating elements among sets of interpretable categories; (4) it is used to conduct formal hypothesis testing; for example, one can evaluate whether an identified gene pattern is significantly different from a null-hypothesis pattern; and (5) it incorporates complex model structures that can be used to exploit external biological knowledge. For example, prior knowledge about expressed genes known to be linked to P53 can be exploited to improve the performance of the present invention, by honing in on the pathways in which they reside.

### EXAMPLE OF THE PRIOR ART

Microarray Analysis of Human Colon Cancer Cell Lines.

Understanding the transcriptional program responsible for tumor metastasis to distant organ sites such as the liver is one key to improving outcomes in colon cancer patients. cDNA microarrays are used to quantify gene expression, and in the present invention is used to identify genes that may play a role in the tumor development and progression, as well as genes that, if differentially expressed, may serve as prognostic markers for patient outcomes. To investigate this transcriptional program, well-characterized sets of matched colon tumor cell lines corresponding to high and low metastatic potential are selected. KM12C is derived from a Dukes' B human colon cancer (non-metastatic). KM12SM is derived from a cecal injection of KM12C with the development of a spontaneous liver metastasis (KM12SM) which upon reinjection into the cecum or spleen results in increased metastatic potential. KM12L4a is the product of 4 passages of KM12C in the nude mouse. Serial intrasplenic injections with harvest of liver metastases are performed, resulting in a cell line with the propensity for liver metastasis upon intrasplenic injection. SW480 is a poorly-metastatic cell line derived from a primary Dukes' C. adenoma and SW620 is a highly-metastatic human colon cancer cell line derived from the lymph node metastasis associated with the primary human tumor.

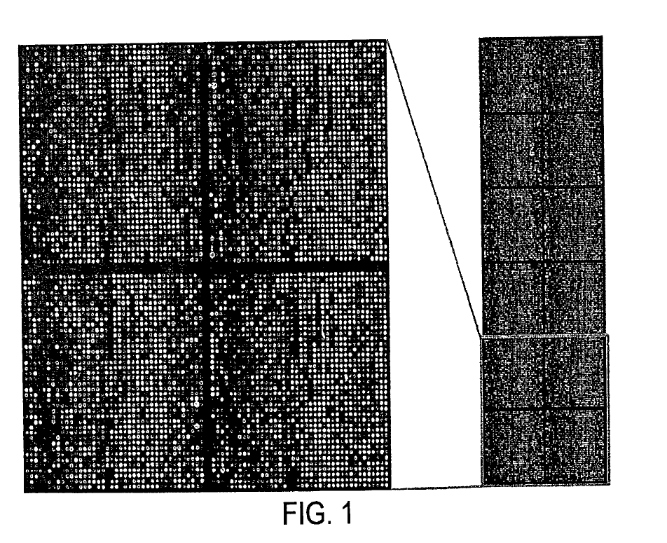

Total RNA is prepared from aloquots of each of these cell lines, and is labeled with probes prepared by incorporation of Cy3 (control=low metastatic cell line, either KM12C or SW480) or CyS (query=highly metastatic cell line, either KM12L4a, KM12SM, or SW620) coupled nucleotides. Probes are cleaned to remove unincorporated nucleotides and hybridized to three replica microarrays containing 19,200 elements. Results of the analysis of an individual pair of cell lines are compared across three replicas to eliminate artifacts due to sample handling or preparation. These arrays are scanned on the General Scanning Scan Array 3000; an example hybridization image is shown in FIG. 1. Images are image analyzed using standard image processing software and integrated intensities for both the Cy3 and CyS probes are measured and stored in a relational database designed to record human microarray expression data. A principal component analysis is sometimes used to enhance small differences in spectral constituents of, e.g., color and/or structure. For details regarding the principal component analysis, see for example incorporated by reference U.S. Pat. Nos. 5,936,731; 5,995,645 and references therein. In essence, any available image processing system takes an input image signal (microarray hybridization spots), then processes to output signal which is stored in a database.

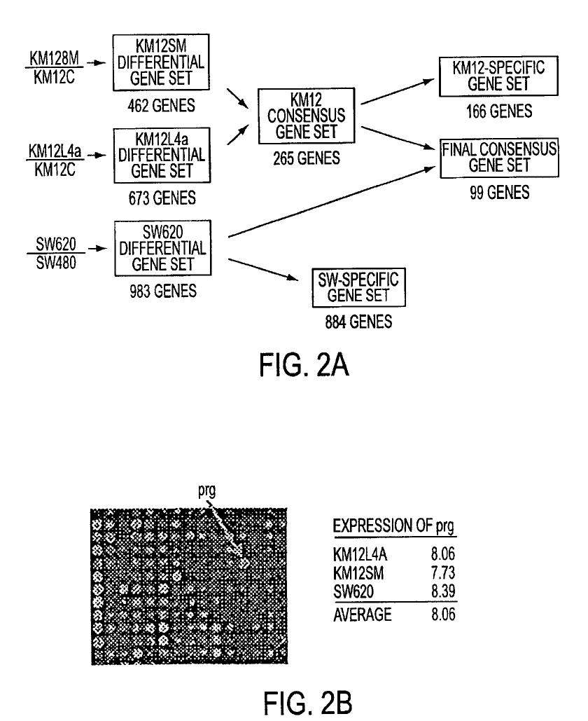

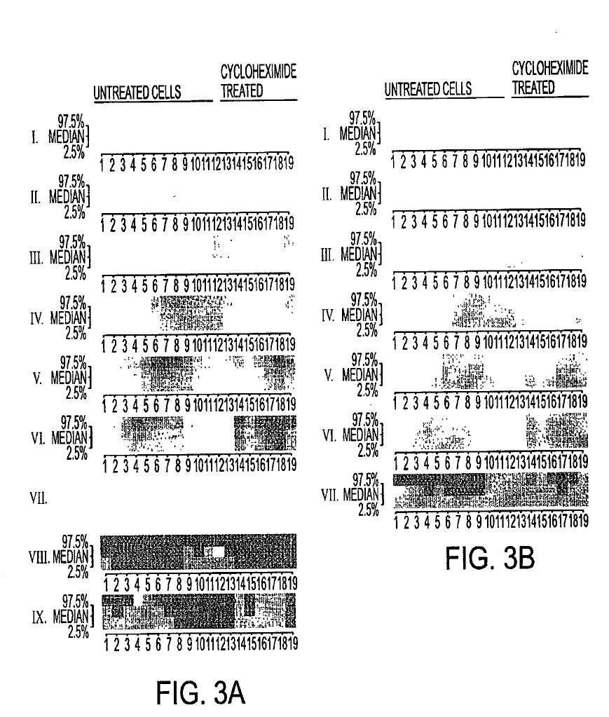

Prior to application of the present invention, fluorescence intensities in the Cy3 and CyS channels are normalized and genes differentially expressed at the 95% confidence level are identified using a variation on the ratio statistics described by Cheng et al. (Cheng, Y., E. R. Dougherty, and M. Bittner, Ratio-based decisions and the quantitative analysis of cDNA microarray images. J. Biomed. Optics, 1997. Vol. 2, pp. 364-374). The Cheng procedure assumes that cellular processes regulate transcript levels such that, for the entire set of genes expressed in a cell, there is a constant coefficient of variation so the variation of the Cy3 and Cy5 signals determined in a hybridization assay is a function of their mean. The normalization procedure that Chen et al., describe uses a set of “housekeeping genes” presupposed to be expressed, on average, at nearly constant levels. This algorithm, as well as a variation that does not make an assumption regarding which genes should be equally expressed in the samples being analyzed but rather allows the data to determine the normalization set, is commonly used. This procedure allows the identification of at least 462 genes that are differentially expressed in KM12SM relative to KM12C, at least 673 genes differentially expressed in KM12L4a relative to KM12C, and about 983 differentially expressed genes in SW620 relative to SW480. Comparison of the gene lists from the KM12 studies produces a consensus of 265 genes. Of these, 99 are expressed in a similar fashion in the SW cell lines. These results are summarized in FIG. 2A. Analysis of the differentially regulated genes presents a biological picture consistent with the current understanding of metastatic progression. Genes found to be up- and down-regulated (shown in parentheses as a fold change) fall into a variety of categories. The growth factors include Cyclophilin A (+2.75), Calcyclin (+2.91); SWI/SNF complex 60 kDa subunit (+2.06); angiogenesis factors such as Platelet-derived growth factor receptor (+1.99); Transforming growth factor beta 1-binding protein (+2.04); Fibroblast growth factor 1 (+2.70). Also seen to be up-regulated are a variety of oncogenes such as Proto-oncogene BMI-1 (+2.14); DJ-1 oncogene (+3.34); Proto-oncogene src-like kinase (+2.64); Stimulatory GDP/GTP exchange protein for c-Ki-ras p21; smg p21 (+2.50). A variety of cell adhesion molecules are up-regulated, while focal adhesion molecules vary in their expression patterns. Protein kinases and phosphatases differentially regulated include Serine/threonine kinase (+2.10); Serine/threonine protein kinase; Krs-1 (+2.17); Serine/threonine kinase STK-1 (+2.29); Adenyl Kinase 1 (-2.92); Tyrosine kinase trkB (-2.77); Protein tyrosine phosphatase (pyst1) (2.53); tyrosine phosphatase, receptor gamma polypeptide (2.98); protein phosphatase, 2A B56-beta subunit (2.19); and Tyrosine phosphatase non-receptor type 1 (-2.53). Apoptosis and cellular proliferation genes are also identified on the arrays, including Tumor Necrosis Factor (Apo-2 ligand) (−2.17); TNF-alpha inducible primary response gene; B94 (2.85); Sphingomyelinase (−2.98); T-cell membrane glycoprotein CD28 (2.34), and IGFBP-5 (−3.60). The genes encoding ribosomal proteins are universally up-regulated indicating that increased expression of genes encoding ribosomal proteins is characteristic of tumor progression. In addition a number of genes of previously unknown function are consistently differentially expressed. An example is shown in FIG. 2B. A gene represented by uncharacterized expressed sequence tags (ESTs), denoted “prg,” is found to be consistently up-regulated by 8-fold across all the analyzed samples. Full-length sequence for the encoded gene shows that it contains a WW protein-binding domain and a leucine zipper, indicating that prg encodes a transcription factor involved in metastatic progression.

### EXAMPLES OF THE PRESENT INVENTION

The practical utility of the approach of the present invention is next illustrated by analyzing three sets of data: (1) The first is data collected in an experiment on fibroblasts that are first serum deprived and then stimulated, to investigate growth-related changes in RNA products over time (see for example Iyer, V. R., et al., The transcriptional program in the response of human fibroblasts to serum. Science, 1999. 283(5398): pp. 83-7 as incorporated by reference); (2) The second represents data derived from the effect of ischemia on the fidelity of microarray expression measurements; (3) The third is a set of colon cancer and normal tissue data (e.g., Alon, U., et al., Broad patterns of gene expression revealed by clustering analysis of tumor and normal colon tissues probed by oligonucleotide arrays. Proc Natl Acad Sci U S A, 1999. 96(12): pp. 6745-50 as incorporated by way of reference). These three actual applications of the present invention are followed by five examples of problems to which the present invention is immediately applicable.

(1) Assessment of Growth-Related Changes in Gene Expression in Fibroblast Cells

In a time-course experiment on fibroblasts, cells are first serum deprived and then stimulated, to investigate growth-related changes in RNA products over time. Other sets are additionally treated with cycloheximide. Samples of untreated and treated cells are collected 1t at 12 and 4 time points, respectively, as are samples of unsynchronized cells. Microarrays include 8613 gene products but present analysis includes about 517 of these. Iyer et al., . Science, 1999. 283(5398): pp. 83-7, used a hierarchical clustering algorithm to identify 10 patterns of gene expression in a subset of 517 genes, which was filtered before application of the clustering algorithm on the basis of the existence of “significant” univariate observed variability or fold changes in gene expression over time.

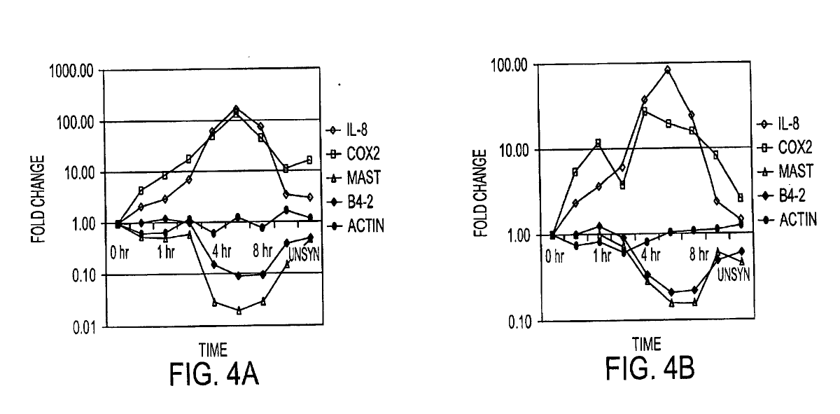

In contrast, the present invention estimates parameters of an intensity-modified homologue of Skene and White's (1992) latent class model for repeated measurements experiments. In this example, the complete set of 8613 gene products is analyzed over all the time points and experimental conditions, employing a normal error model on the log-transformed data, with mean conditional on latent gene class and time (plus experimental condition), with gene-specific intensity modifiers to represent the degree to which each gene is a good marker of its associated latent gene class. This approach uncovers about 9 time-correlated patterns. Genes have either decrease in expression (negative intensity modification parameters) or increase in expression (positive intensity modification parameters). FIG. 3 presents both the estimated patterns and their inverses. In other words, red and green colors represent, respectively, higher or lower levels of expression relative to untreated cells at time 0. A white background aids visualization; brighter colors represent greater relative deviation from baseline. In addition, the invention adjusts for the data quality in the underlying microarray data. Specifically, an observation (one spot on one microarray slide) is treated as missing whenever the inter-pixel correlation between the two scans is less than 0.6. Thus, the invention accounts for and is unbiased by differential hybridization of samples. A consequence of this is that Pattern VIII is distinct from Pattern VI because of insufficient information at samples 1, and 9 through 13, as evidenced by a wide confidence interval ranging from very bright green (2.5%-ile) to very bright red (97.5%-ile). Probabilities of gene membership in these latent patterns for every gene in the data are estimated by the model. Estimated probabilities for some of the elements of selected patterns are given in Table 1.

TABLE 1

| Pattern | Expressed Gene | Probability |
|---|---|---|
| VI | Interleukin-6 Precursor | 100% |
| | P55-C-Fos Proto-oncogene | 100% |
| | Human TGF-Beta Inducible Early Protein | 100% |
| | Myc Proto-oncogene protein | 100% |
| VIII | Transcription factor Jun-B | 100% |

This example illustrates some additional advantages. Estimated time-course patterns are smoother than those resulting from hierarchical clustering analysis, even though no smoothness criterion is imposed by the instant model. Although no assumptions are made regarding the correlations within treatment group across time, the estimates show expression patterns that are correlated with known stages of cellular growth and mitosis, indicating that the instant model is uncovering the underlying biology. No prefiltering of genes is required by instant technique. Data quality issues are adjusted by using statistical approaches, to reduce the potential biases that can be introduced by experimental variability, especially at low spot intensities. Standard statistical approaches to diagnose lack-of-fit of instant model to the data are also feasible.

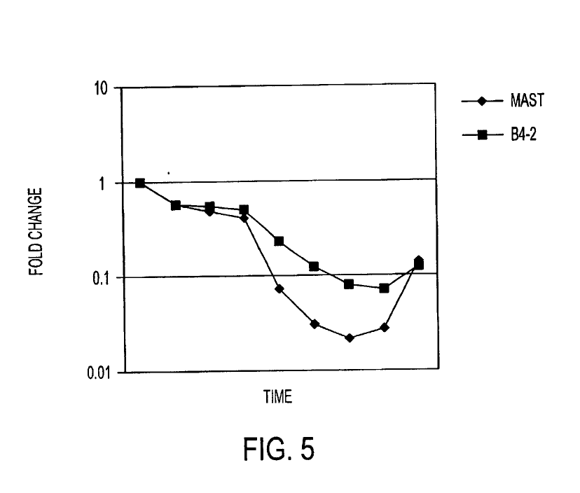

The invention makes it possible to adjust for known issues with microarray estimation of true expression relative to Northern blot experiments. By way of illustration, the published TaqMan™ (RT-PCR) assay and microarray results of the above fibroblast data for 5 expressed genes are reproduced in FIG. 4. Although there is substantial correlation between the results, they are significantly different. Specifically, the microarray data are more variable across the experiment, underestimate large changes in RNA expression, and suffer from edge effects. All three problems are illustrated by the Mast and B4-2 genes. For example, the TaqMan™ assay provides that there are substantial differences in expression between Mast and B4-2 from 4 hours on, at which time Mast is an order of magnitude more repressed than B4-2. Because the present invention pools information across gene products and microarray hybridizations, it uses biological correlations across measurements to reduce biases in particular observations. The present invention also adjusts for biases inherent to specific biological techniques.

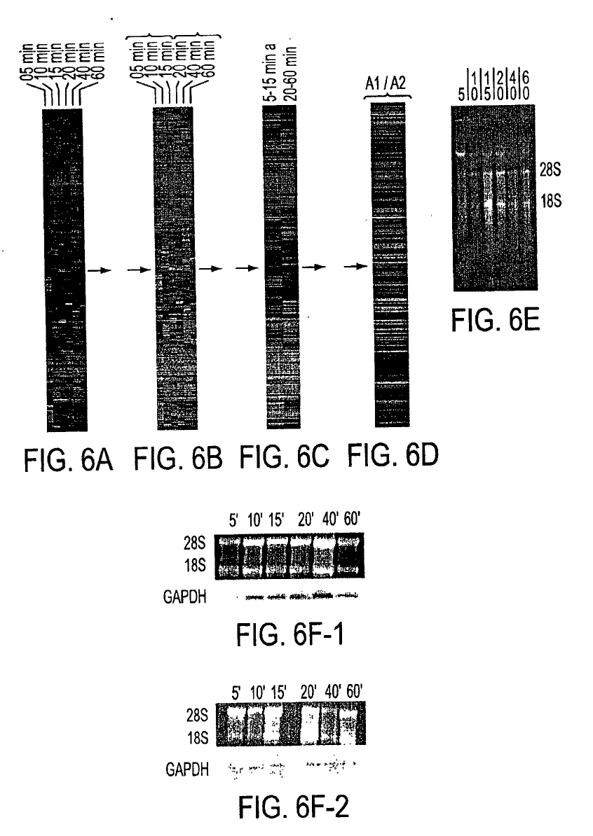

FIG. 5 shows adjusted SPAM estimates of Mast and B4-2 expression derived from the microarray data. This approach is easily adaptable to other situations such as for example analyzing changes in expression of fibroblast and epithelial cells in response to selenomethionine, a compound known to decrease clinical risk of certain cancers in high-risk groups. Data from Northern blot and microarray data can be analyzed using the present invention to verify and adjust expression quantitation.

To characterize and control the intrinsic variability associated with the microarray process, KM 12 human colon cancer cell lines are used as a renewable source of control RNA. To estimate the number of repetitions required for consistent estimation of a single sample's gene expression pattern, replicate microarray analyses are tested. Initially, this presents as hybridization of as many as 20 chips across different RNA and probe preparations, and across-chip construction runs of the microarray (up to 100 slides are produced per run). The within-gene between-run variabilities are compared and information obtained is used in refining the analysis. These analyses identify steps in the microarray process that are highly variable, and can be controlled through alterations in procedure. These variabilities provide an important benchmark for quality control.

(2) Ischemic Effects on the Fidelity of Microarray Expression Measurements

The degradation of RNA in tissue samples following excision from the patient can have a significant effect on the expression measurements that are derived from microarray analysis. If RNA species degrade at different rates, the relative population frequency of a particular species will change over time. A microarray comparison of RNA levels between identical samples subjected to different ischemic times could lead one to the incorrect conclusion that some of the genes on the array are differentially expressed. For example, consider a particular tissue type in which there are only two RNAs present, RNA A and RNA B, and that these RNAs are expressed at the same level in normal tissue. However, RNA A is stable while RNA B decays with a relatively short half-life. Now, as a reference sample an amount of RNA equivalent to 1000 molecules is extracted; that sample contains, on average, 500 molecules each of RNA A and RNA B. When a second RNA sample from the tissue is extracted at some later time the level of RNA B are fallen to 20% of its starting value. At this time, the total number of RNA A molecules is unchanged, but the number of RNA B molecules is only ⅕ of its starting value. Consequently, the ratio of RNA A and RNA B is changed. Specifically, the same total mass of RNA from this later sample—1000 molecules—will consist of ⅚ of RNA A (833 molecules) and ⅙ is RNA B. If this sample is compared to reference sample, it would appear that RNA A had been up-regulated by 1.7-fold and RNA B had been down-regulated by nearly 3-fold. If one compares two different patient samples without prior knowledge of the RNA stabilities or of the ischemic time the samples were subjected to, one concludes that these RNA species are differentially expressed. This suggests that the handling of patient samples is crucial for generating meaningful expression data using microarrays in order to avoid reaching spurious conclusions regarding expression levels.

In order to test this occurrence the RNA levels are measured in a patient-derived tissue sample following different periods of warm ischemia. The goal is to see if the observed RNA levels might mimic results that would be obtained from a differential expression experiment. Normal colon tissue removed during a bowel resection is divided into small sections immediately following excision. Tissue segments of approximately equal size are placed into liquid nitrogen at 5, 10, 15, 20, 40, and 60 minutes following removal. Total RNA is extracted using Trizol and RNA samples are used for microarray expression analysis. Poly(A+) RNA is prepared from each using oligo(dT) coated Seradyne magnetic beads and labeled oligo(dT) primed first-strand cDNA probes are prepared by incorporation of CyS-dUTP. A reference probe is prepared and labeled with Cy3-dUTP using an equal quantity of mRNA extracted from the KM12C cell line.

For each time point, labeled cDNA from the clinical sample and from the cell-line control are co-hybridized to a microarray containing at least 2,400 distinct elements. Hybridized arrays are washed and then imaged using the ScanArray 3000 confocal laser scanner. TIFF images produced are analyzed using TIGR Spotfinder and background-subtracted integrated intensities for each spot are recorded. For each experiment, signal intensities between the two fluorescent images are normalized by separately summing the intensities in each channel and scaling the Cy3 intensities so that the summed intensities for both channels are equal. This normalization approach relies on the assumption that the same total quantity of RNA is used for both the query and control samples. For each of the approximately 2400 arrayed genes, the natural logarithm of the ratio of Cy5/Cy3 background-subtracted intensities is determined and used for subsequent analysis. Gene and time-point cluster analysis is performed using the Cluster/TreeView hierarchical clustering package (see incorporated reference Eisen, M. B., et al., Cluster analysis and display of genome-wide expression patterns. Proc Natl Acad Sci U S A, 1998. 95(25): pp. 14863-8), which uses Pearson correlation coefficients as a measure of similarity and average-linkage clustering. Clustering results are displayed visually, with each experimental time-point represented by a t column in the display and each gene by a separate row. Elements of the display are colored to represent its mean-adjusted ratio value; red-colored and green-colored cells represent, respectively, higher and lower levels of expression relative to the test sample; relative expression is represented by the relative brightness of the signal.

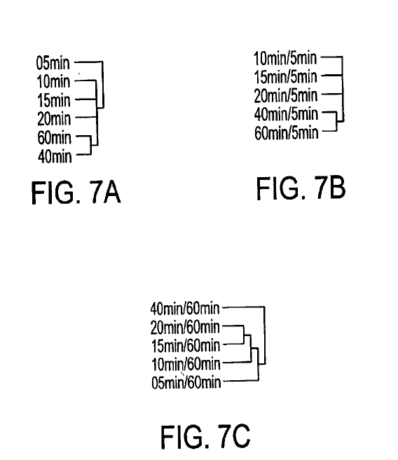

Of the 2,400 genes in the arrays at least 2,114 provide useful data in at least one experiment. Analysis of the 1,420 genes that gave signals above background for all eighteen measurements is performed. A time course, with genes clustered by temporal expression pattern are shown in FIG. 6. In each colored figure, the temporal expression pattern of each gene is represented by a horizontal row; the expression patterns of all genes in a single experiment is represented by a column. Using this red/green display, it is difficult to visually assess relative changes in gene expression levels over time. However, FIG. 6(C), represents the gene expression levels averaged from 5-15 min next to those averaged from 20-60 min to demonstrate visible differences over time. These differences are further emphasized in FIG. 6(D) where the ratio of these averaged gene expression levels is displayed and any color other than black represents a change in expression over time. These alterations in gene expression linked to ischemia over the course of 60 min are not visualized in the ethidium bromide gel analyses and/or Northern analyses for a single housekeeping gene (FIG. 6E and F). Of the genes analyzed 45.4% (644 of 1420) show an increase in expression, 28.2% (401) show a decrease, and the remainder (26.4%; 375 of 1420) show a more complex temporal pattern of expression. Analysis by Eisen's clustering analysis does identify some relationships suggesting that the earlier time points cluster away from later time points (FIG. 7); however, the method gives no indication as to the reason for the clustering.

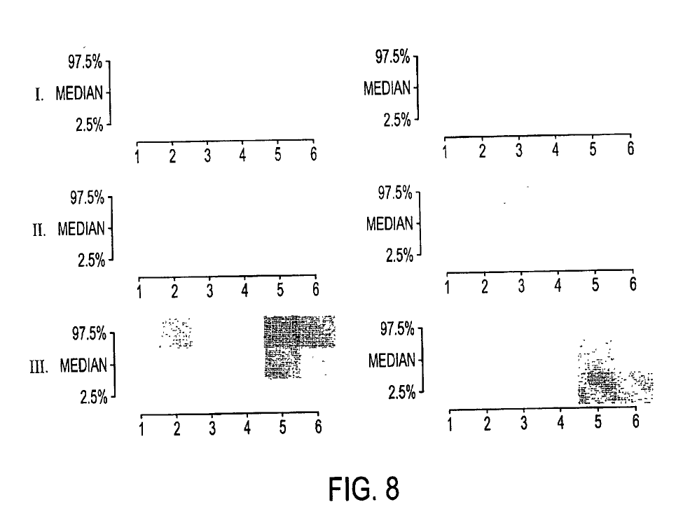

Application of the time-course model using the present invention reveals that there are 3 patterns in the ischemia data, as shown in FIG. 8. These three prevalent patterns account for 68.2%, 17.8% and 13.4% of the 1420 genes, respectively. Pattern I corresponds to an average change of 27% over 60 minutes from 5 minute baseline level of expression. 63.8% of the genes with at least 80% probability of membership in this pattern show average to increases in expression over 60 minutes (left panel). The remainder decrease on average (right panel). Pattern II genes show the least ischemia-related effects, demonstrating an average change of only 12% over 60 minutes. In contrast to pattern I, 67.5% of the genes with at least 80% probability of membership in this pattern are decreasing in expression on average over time (right panel). The remaining 32.5% in this pattern increase an average of 12% over 60 minutes. Finally, pattern III genes (13.4% of the sample) show the greatest sensitivity to ischemia, changing an average of 50% over 60 minutes, with about the same number increasing as are decreasing. In all these patterns, the null hypothesis of no change is not rejected with small sample size at any of the first 4 time points, from 5 through 20 minutes. In patterns I and III especially, 40 and 60 minute time points are found to deviate significantly from the 5 minute expressions. This is evidenced in the figure by confidence intervals that do not contain the 0 change or 1.00 relative expression ratio (white or no color) for averaged samples 5 and 6. It is obvious that more data and the conduct of more refined time-course experiments, will allow one skilled in the art to determine both what genes are most susceptible to the effects of ischemia, and to design a microarray test that will grade tissue samples according to extent of ischemic degradation. Such an approach is extremely useful in adjusting data derived from tissue bank samples.

Based on these data, a number of clear conclusions are drawn. First, temporal changes in gene expression levels do occur following tissue excision, with detectable changes after as little as 20 minutes. This observation is in conflict with the conclusions one might draw regarding the “quality” of the RNA based on 18S and 28S bands seen on a gel (FIGS. 6E and 6F). Typically, stability of RNA is usually determined by the integrity of the 118s and 28s ribosomal bands on an agarose gel. The gel shows a good 18s:28s ratio for tissue RNA samples obtained at all time points, which suggests that the RNA is stable. However, microarray analysis of the same RNA samples clearly shows that there is significant degradation of RNA over time during ischemia. Thus, using the 18s:28s ratios as a measure of RNA stability is misleading. Regardless of whether these changes occur due to actual cellular processes or if they are the result of RNA degradation during ischemia, they produce significant and fundamental changes in the relative representation of RNA species. Consequently RNA samples compared from the same patient sample held for different times at room temperature following excision will exhibit patterns of differential expression that may be confounded with research questions, regardless of actual patterns of gene expression in vivo. These results suggest that unless tissue samples are carefully handled and snap frozen in an expedient manner, expression measurements are likely to be highly suspect.

(3) Molecular Fingerprints of Colon Cancer and Normal Tissues

Colorectal cancer is a common, deadly disease with 129,400 new cases and 56,600 deaths projected for 1999 in the United States. While surgical resection of localized tumors may be curative, the vast majority of deaths are linked to the metastatic spread of tumor cells. Sporadic colorectal cancer is known to arise from an accumulation of multiple, sequential somatic genetic changes within a cell, each of which likely has complex effects on gene expression. This invention addresses the problem of identifying molecular fingerprints relating to colorectal cancer metastasis as a means of substantially improving diagnostic and prognostic capacities, and potentially elucidating new mechanisms underlying the metastatic process Sporadic colon cancer is the result of multiple, sequential somatic genetic alterations, which likely affect numerous pathways. Early epidemiological studies predicted that at least 5-6 genetic events would be required to generate a colon cancer. It is now appreciated that somatic mutations in the APC gene are common to the vast majority of colorectal cancers. Its mutation is the first step towards carcinogenesis, a step that leads to a multitude of complex downstream pathway effects. Its alteration often leads to truncation of its product, with subsequent downstream effects on multiple APC partners including catenin, p130Cas, E-Cadherin, and T cell factor-4 (Tcf-4). More specifically, it has been determined that mutations in APC or in catenin increase the activity of the catenin/Tcf-4 complex, leading to overexpression of c-MYC and cyclin D1 with subsequent promotion of neoplastic growth. APC mutation can now be related to the downstream effects of c-MYC on gene transcription and translation. For example, recent studies have demonstrated that MYC activities are modulated by a network of bHLH-Zip proteins with MAX at the center of the network. Whereas MYC-MAX complexes activate transcription, MAD-MAX complexes repress transcription. For this reason, mRNA/protein levels of critical genes may increase or decrease in response to specific upstream stimuli. Like APC mutation, mutation of RAS is also thought to be an early event with downstream effects on signaling pathways involving many partners including Raf, MEK, and MAPK. Recently, Ras has been implicated in the Myc pathway by the finding that Ras enhances the accumulation of Myc activity by stabilizing a protein with an otherwise short half-life. Subsequent to APC and/or RAS mutations, genetic events associated with tumor progression are thought to include the alteration of genes such as DCC, DPC4, and P53. The instant inventors have recently described a mutation of SRC in codon 531 which may contribute to the aggressiveness of advanced tumors with metastatic potential. Each of these somatic genetic events burden affected cells by triggering multiple downstream changes in gene transcription and translation, thereby increasing the capacity of the cell to progress and develop deadly metastatic potential. The challenge is to identify the critical components of each pathway affected by these genetic alterations and to characterize the network connections.

Metastasis is a common problem linked to the altered expression of numerous genes. Metastasis is thought to be an evolutionary process based on the generation of tumor cells, which have accumulated a defined set of biological capacities through mutational events. These include the capacities to develop new blood vessels, to grow, to invade protective basement membranes, to detach, to clump and form emboli, to evade host immune systems, and to attach and grow at distant organ sites. Failure to develop one or more of these capacities results in the elimination of metastatic potential. To date, numerous molecules have been implicated in models of metastatic progression. These include angiogenesis factors such as VEGF, invasive enzymes such as metalloproteinases, collagenases, and heparinases, adhesion molecules such as integrins , cadherins, catenins, and annexins, and cell surface glycoproteins like CEA. Interestingly, the majority of molecules linked to the metastatic cell represent a sometimes subtle, over- or under-expression, of what is already expressed by the normal cell. The evolution of metastatic potential occurs within the primary tumor. The process is the result of linked, sequential mutational events, whereby numerous phenotypic traits must be altered in some orderly fashion. It is now widely accepted that malignant tumors contain heterogeneous subpopulations of cells with significant biologic differences. Whereas the majority of cells in the primary tumor may have the genetic expression patterns predisposing to metastasis, these traits alone may be insufficient to produce a metastatic cell. The capacity of a tumor to metastasize is attributed to smaller subpopulations of cells, pre-existing within the primary tumor, which have both the predisposing and essential traits permitting distant spread. This model may be an oversimplification, but it does describe why the metastatic process is considered inefficient, with less than 0.1% of the primary tumor cells being capable of distant spread. While essential traits may only be found in small portions of the primary tumor, we believe that the predisposing traits, which precede the development of essential traits, are present in the majority of the tumor and will be prevalent enough to target and decipher. Current staging systems based on anatomic descriptions are inadequate to predict metastasis. There are several clinicopathologic staging systems currently in use which are based solely on anatomic descriptions of tumor and the degree of tumor spread. The oldest system, the Dukes' staging system, delineates tumors into four groups (A,B,C,D) based on histologic evidence of tumor progression. Dukes' A tumors are node negative and involve only the mucosal and submucosal layers of the bowel wall. Dukes' B tumors invade deeper into the bowel wall involving a portion of, or complete penetration through, the smooth muscle layer. Dukes' C. tumors metastasize locally to the draining regional lymph nodes and Dukes D tumors metastasize distantly to organs such as the liver and lungs. Despite the relative effectiveness of current staging systems, they do not incorporate prognostic variables such as differentiation, lymphovascular invasion, or clinical complications such as tumor perforation or fistula formation. Moreover, no genetic pathway information is utilized. Even well-studied single genes, such as P53, have yet to gain clinical favor, presumably because of their inability to significantly improve upon the power of current staging systems. With the introduction of high-throughput microarray and proteomic technologies, however, new staging systems may incorporate extensive molecular data that differentiate fingerprints for tumor diagnosis and behavior. One testable hypothesis is that some of these differential fingerprints are directly related to the phenotypic (histologic) differences among tumors that permit differential recognition by pathologists. Other fingerprints may provide powerful prognostic information, but result in no visible phenotypic differences. More precise molecular staging would assist clinicians in better identifying the subsets of patients who might benefit from a specific therapeutic intervention while at the same time providing insight into the mechanisms underpinning the metastatic process.

Microarray expression analysis is used to analyze differential gene expression patterns. For the general notion of gene expression analysis by using microarrays several well-known references exist in the art as enclosed herein by way of reference. The principles of such technologies are disclosed in numerous U.S. Pat. Nos. such as 5,556,752, 5,744,305, 5,837,832, 5,843,655, 5,874,219, 5,849,486 and PCT patent publications such as WO 99/27137 and WO 99/10538 all of which are incorporated by reference herein.

Many different types of microarrays thus exist which are all equally suitable for analysis by the present invention. In what follows, the new method is used to analyze published data on colon cancer and adjacent normal tissues, to demonstrate the ability to simultaneously identify and quantify molecular fingerprints that differentiate cancer from normal tissues, as well as latent gene classes as described supra. The data consist of gene expressions in 40 tumor and 22 adjacent normal colon tissue samples, that are derived from a commercial Affymetrix oligonucleotide array complementary to over 6,500 genes.

The invention employs two-dimensional models with forms like log(Yᵢⱼ)|iεSₘ, jεGₗ˜|tᵢₗ (αₘₜ, βᵢⱼ, γₘₗ), σ²|, where i and j index the expression data by gene and sample respectively, m and 1 index latent classes on the corresponding dimensions, the t refer to gene-specific intensity parameters, and various forms for the function f are chosen. The following analytic results are based on an additive form for f, αₘₜ, βᵢⱼ, γₘₗ, that is, an ANOVA-like additive model is used for incorporating main effects and an interaction term for the latent class means. Using its complete conditional distributions in the presence of weak prior information, a modified Metropolis algorithm is employed to converge to draws from the posterior distribution of this model. No information about the known cancer classification of the tissues is provided to this model, since the objective is to illustrate the SPAM model's ability to differentiate and quantify molecular fingerprints of differing tissue types.

As a result, evidence is obtained to support the existence of up to 5 latent tissue classes in these data. Two of these classes contain relatively large proportions of normal or cancer tissues. The estimated assignment of each tissue into each latent tissue class is given in Table 2. Tissues are assigned based on having a greater than 80% probability of membership in a particular class. The present invention identifies prevalent tissue classes that consist primarily of normal or cancer tissues, in latent tissue classes I and II respectively. This is confirmed by Fisher's exact tests indicating that latent tissue classes I and II discriminate between normal and cancer tissues (p<0.0001).

TABLE 2
Assignment of 62 Normal and Cancer Tissues Among the Five Classes

| | Latent Tissue Class | | | | |
|---|---|---|---|---|---|
| | I | II | III | IV | V |
| Cancer | 1 | 30 | 5 | 2 | 2 |
| Normal | 16 | 2 | 3 | 1 | 0 |

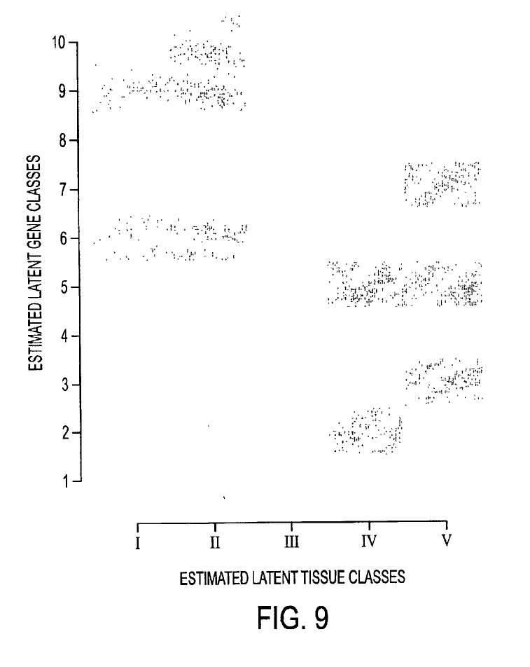

The latent grouping of normal and cancer tissues uncovered in this analysis results from gene expression patterns across tissue samples. As in the two analyses presented above, genes are estimated to belong to each of a set of latent gene classes. In this analysis, 10 latent gene classes are found, each of which corresponds to a set of genes that express similarly both within and across tissues. In other words, genes in a single gene class would all tend to be elevated in the same tissues, or alternatively, repressed in the same tissues. The interaction between latent gene classes and latent tissue classes, as shown in FIG. 9, illustrates the ability of certain gene classes to discriminate among the estimated tissue classes. For example, this figure demonstrates that expressed genes that are primarily in latent gene class 7 are upregulated in most normal tissues relative to most cancer tissues in this data set; however, they are also highly upregulated in tissue classes III, IV and especially V, which have tumor as well as normal tissues. The latent tissue class III has many relative expression levels between those of tissue classes I and II, implying that it may consist of earlier stage cancer tissues and adjacent normal tissues with pre-malignant changes in gene expression.

This analysis illustrates the potential complexity of a molecular fingerprint that can discriminate among potentially interesting tissue classes. For example, use of a single gene in latent gene class 7 is likely to result in less powerful discrimination among tissue types than would the use of a few genes in each latent gene class. Taken together, a set of genes extracted from each of the informative gene classes can be used to generate a practical indicator of a tissue's molecular fingerprint, that can be applied to a dedicated microarray chip. For example, a set of gene culled from gene classes 1, 4, and 7 can together better discriminate between normal and cancer tissues than can any single gene or single gene class alone. The present analysis allows one to select this set of genes, by identifying the genes that have the highest estimated intensity within each gene class, and therefore, these genes would be most useful as markers of the associated latent gene classes.

With the caveat that looking at individual genes is insufficiently informative to distinguish among tissue classes, a series of genes in each of the estimated latent gene classes are considered, and as a result interesting results are obtained. For example, IGF binding protein, proto-oncogene RET precursor, and EGFR are all in latent gene class 1, meaning that the expression of these genes is elevated in most tumor tissues relative to normal. Similarly, latent gene class 4 contains genes relatively overexpressed in most tumor tissues that encode for transcription factors, DNA binding proteins, ribosomal proteins and ataxia-telangiectasia, all of which are linked to neoplastic processes. Muscle genes considered discriminating and perhaps related to the existence of more contaminating muscle tissue in the normal colon samples, are found in latent gene classes 3, 6, 7 and 9, all of which are upregulated in the normal tissues. These finding concur with the added value of differentiating between muscle genes that are differentially expressed between most normal and cancer tissues (latent gene classes 3 and 7) versus those that are upregulated in both tissue types (latent gene classes 6 and 9).

These analyses demonstrate that a prognostic fingerprint can be identified by the novel method to select patients with, for example, liver metastases who could be cured from a resection procedure. Liver resection is potentially curative in up to 30% of patients selected, based on the number of colorectal liver metastases being <4, the location being generally unilobar, and the absence of porta hepatis lymphadenopathy. Despite being carefully selected for resection, there is currently no way to identify which 30% of the resected patients will be cured.

(4) Deciphering Comprehensive Proteomics Data

“Proteomics” is a recently coined term to denote the use of quantitative protein level measurements of gene expression to characterize biological processes and decipher the mechanisms of gene expression control. While gene expression may be directly linked to mRNA levels within the cell, it is not always the case that mRNA levels predict protein levels. In fact, it has been reported that protein and mRNA abundances may correlate poorly, with a correlation coefficient as low as 0.48, secondary to mechanisms of post-transcriptional and post-translational modification. For this reason, the comprehensive evaluation of protein expression may be equally as important as the evaluation of mRNA expression. Proteomics analysis is performed by combining 2D-gel electrophoresis (2D-GE), to separate and quantify protein levels, with two forms of mass spectroscopy to identify selected proteins of interest within the 2D gel. 2D-GE is the highest resolution analytical procedure for routine global analysis of proteins currently available, and it is feasible to do large-scale quantitative protein mapping studies, albeit in only a few specially equipped laboratories worldwide. A number of non-quantitative or semi-quantitative 2DE studies have been done to attempt to find exploitable differences between normal and tumor cells, and to develop databases of the protein composition of tissues including liver, brain, heart, keratinocytes, and blood proteins, among others. 2D maps of human plasma, urine, saliva, milk, semen, human lymphocyte proteins, human lens, and proteins of rat tissues are now available.

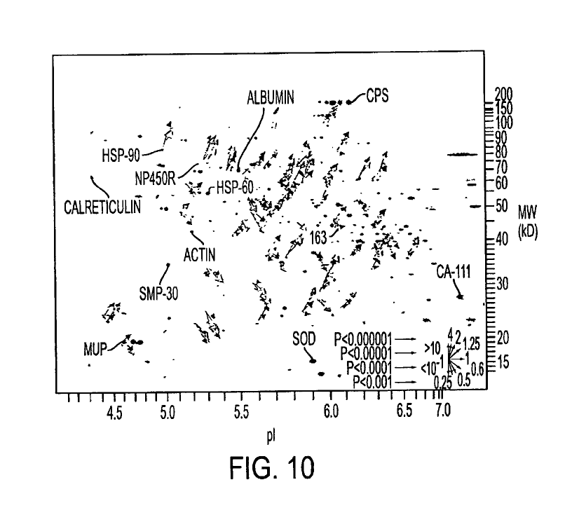

The principles of 2-D gel analysis of proteins are well established, e.g., Anderson, N. L. and N. G. Anderson, Analytical techniques for cell fractions. XXII. Two-dimensional analysis of serum and tissue proteins: multiple gradient-slab gel electrophoresis. Anal Biochem, 1978. 85(2): pp. 341-54; Anderson, N. G. and N. L. Anderson, Analytical techniques for cell fractions. XXI. Two-dimensional analysis of serum and tissue proteins: multiple isoelectric focusing. Anal Biochem, 1978. 85(2): pp. 331-40. 2-D gel databases of proteins are commercially available from specialized companies, e.g., Large Scale Biology (LSB). LSB's Molecular Effects of Drugs™ (MED™) and Molecular Anatomy and Pathology™ (MAP™) databases contain more than 10 million protein abundance measurements. The MED™ database, for example, already contains 2-D gel information characterizing the effects of almost 100 chemical agents (mostly pharmaceuticals) on the protein expression pattern of rodent liver in vivo, and allows a molecular approach to the investigation of toxic and therapeutic mechanisms. The MED™ database is currently being expanded to include proteome analyses of more than 50 different human tissues. These databases provide master gels which are useful in the identification of proteins from unknown tumor specimens through gel matching techniques. The LSB Kepler system involves an extensive two-dimensional mathematical filter that removes background, deconvolves each protein spot into one or more Gaussian peaks, and calculations the volumes under each peak (representing protein quantity). A multiple montage program allows the comparable areas of a series of up to 1,000 gels to be displayed and inter-compared visually to check on pattern matching. In matching individual gels to the chosen master 2-D pattern, a series of about 50 proteins is matched by an experienced operator working with a montage of all the 2-D patterns in the experiment (see FIG. 10A for a representative 2-D gel of proteins after exposure to peroxisome proliferators). Subsequently, an automatic program is used to match additional 600-1000 spots to the master pattern using as a basis of the landmark data entered by the operator. Because a 2D-GE analysis of an individual tumor results in a protein molecular fingerprint which is directly compared to that of numerous other tumors, differentially expressed proteins are rapidly identified. For the sequence identification of differentially-expressed proteins, a PerSeptive Biosystems Voyager DE™ STR BioSpectrometer Work Station is used, which achieves mass accuracies of <50 ppm and usable sensitivities of 7 femtomole peptide applied to the target. Instruments such as Finnigan LCQ ion trap mass spectrometer and Michrom Magic 2002 microbore HPLC are used for generation of protein sequence from 2-D gel spots via LCIMS/MS methods. Proteins from 2-D gels are excised and used to make antibodies (polyclonal or monoclonal) according to standard procedure well known in the art, which are then used in immunohistochemical analyses to examine the location of the protein within a tissue or within a cell.

Analysis of complex quantitative differences among a series of protein expression patterns has advanced significantly over the last several years. A series of treated or diseased samples can be compared quantitatively, and abundance ratios (treated or diseased divided by normal control values) can be calculated. Subsequently, protein spots are selected that show differences among the groups. Such results are plotted, and multiple comparisons are made for consistency (FIG. 10). In the specific instant case a series of drugs known to be non-genotoxic liver carcinogens in the mouse, are compared and found to produce consistent effects on the abundances of a large series of identified liver proteins, with concordant increases or decreases. This approach is exploited to examine molecular fingerprints that are shared between a primary tumor and its paired metastasis. Because proteomic analyses are capable of examining serum proteins, a differential analysis of patient-derived serum samples is carried out to look for secreted proteins linked to the process of metastasis.

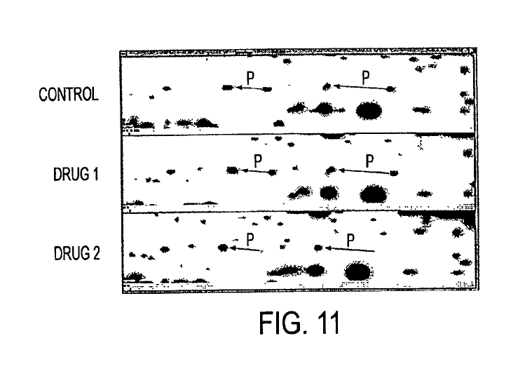

Moreover, with the elucidation of several critical signal transduction pathways, such as the Ras pathway, it is now clear that not only gene expression, but also phosphorylation pattern of gene products, is central to the regulation of the cell and a critical part of the comprehensive analysis of gene expression. As phosphorylated and unphosphorylated versions of a protein occur at different locations on a 2-D gel, differential quantitation of the forms is further assessed (FIG. 11).

(5) Deciphering Pathways Related to a Particular Gene

P53, a tumor suppressor gene, is the most commonly mutated gene in human cancer, being mutated in up to 80% of colorectal cancer, and is likely involved more in tumor progression than initiation. The majority of the mutations are point mutations, which occur in codons 5-8, and result in single amino acid changes in the protein. Human cancers frequently contain an allelic deletion of the P53 gene, a deletion which is found in up to 70% of colon cancers. In addition, P53 has been found to contain germline mutations. In each of these cases, a somatic mutation of the remaining normal P53 allele may lead to sporadic cancer, as a result of altered or absent P53 protein activity. The P53 protein is involved in cell cycle checkpoints in both G1 and G2. When DNA damage occurs, P53 levels increase, at least in part, because of stabilization of the existing protein by phosphorylation. Active P53 then induces one or more of the following events: cell cycle arrest, DNA repair, and apoptosis. In addition, evidence shows that there may be a gain of function in mutant P53, likely affecting downstream gene expression. Analysis of DNA binding sites for P53 suggest that there may be a hundred or more genes, as yet unknown, regulated by P53. For a general information regarding p53 and cancer oncogenes see for example incorporated U.S. Pat. Nos. 5,620,848, 5,527,676, 5,998,136 5,983,211, 5,747,469, and references therein. Elucidation of the downstream effects of mutant P53 in cells will aid in the identification of possible targets for drug discovery. Metastatic tumors frequently harbor mutated P53 and gene expression patterns identified in cell lines with experimentally induced mutant P53 are comparable with human tumors known to contain mutant P53. These patterns are useful to decipher metastasis-specific gene expression patterns by identifying P53 linked gene expression patterns, or portions thereof, in tumors with documented metastatic behavior.

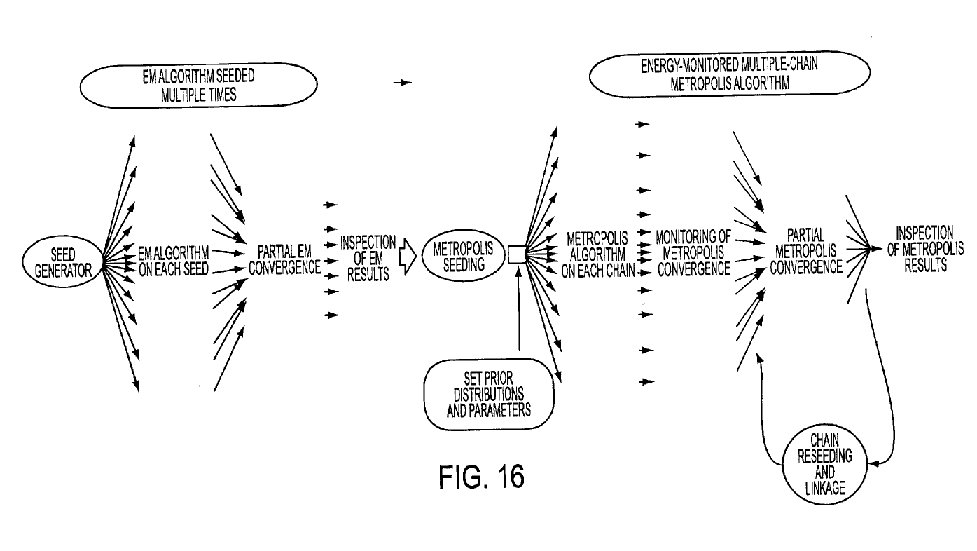

The gene expression patterns are produced by the overexpression of wild-type P53 by using human colon cancer cells with a wild-type, endogenous P53 (HCT 116) transfected with the V138 temperature sensitive mutant. At 32° C. the mutant P53 does not disrupt wild type (wt) P53, thus MDM2 and p21 WAF are induced. At 39° C. degrees, the mutant P53 disrupts wtP53 and results in inactivation of MDM2 and p21WAF as shown in FIG. 16 (for details on p21WAF and MDM2 see U.S. Pat. Nos. 5,807,692; 5,858,976; 5,770,377; 5,756,455; 5,721,340; 5,708,136; 5,702,908; 5,702,903; 5,693,533; 5,550,023 as incorporated herein by way of reference). As there is a large induction of MDM2 and P21WAF1, one skilled in the art can use this information to validate the microarray analysis, because both genes are represented in the instant array. Thus our approach is valid and is readily adaptable to the exploitation of the instant data with publicly available data concerning the analysis of cells transiently transfected with wild-type P53. It is anticipated that by analyzing as many as 7,202 transcripts at least 14 are over-expressed at levels 10-fold greater than controls. By using microarray analysis setting appropriate parameters with considerably larger gene sets, more genes are likely to be obtained.

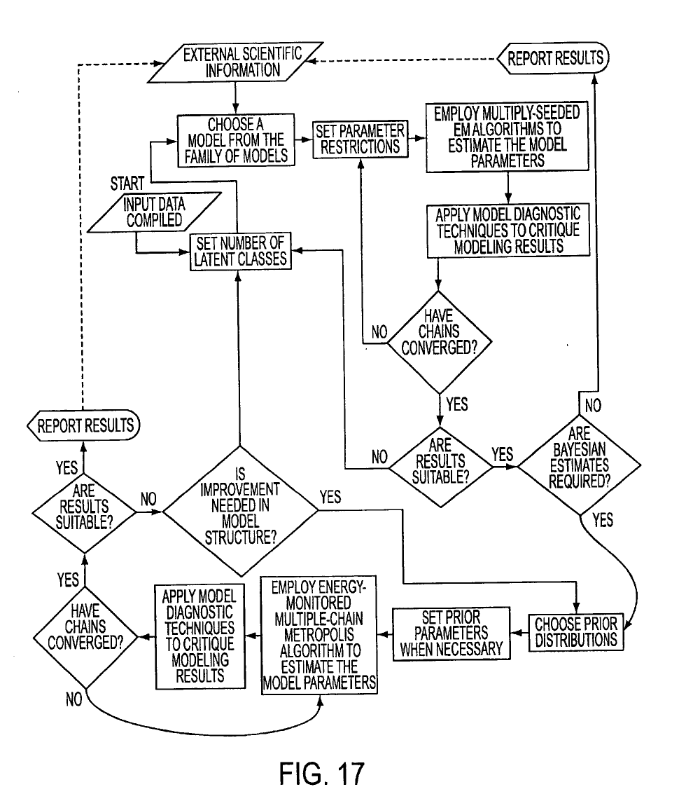

In addition to the instant V138 transfectants at 39° C. as a model other models of a cancer cell are equally suitable, e.g., transfected HCT 116 cells with a construct that expresses HPV E6, a protein known to target endogenous P53 for degradation. Critical differences in gene expression patterns between cell lines with over-expressed P53, wild-type P53, and suppressed endogenous P53, are thus addressable using either microarray or proteomic analyses. The patterns derived from these analyses are then compared with those from panels of cell lines with known P53 mutation (e.g., SW480, DLD-1) or wild-type P53 (LS180, HCT116) to determine their capacity to model P53 mutation in human colon cancer cells. As next logical step one skilled in the art will know whether these patterns are applicable in human tumors specifically harboring a mutant P53 gene versus those found to be wild-type for P53. And finally, these patterns induced by P53 mutation are directly comparable with gene expression patterns produced by tumors known to be metastatic to determine if strong correlations or shared gene expression patterns exist. This mathematical approach is reduced to practice as illustrated by FIG. 17.

(6) Characterizing a Drug Effect.

Human hepatoma cell lines, Hep3B and HepG2, are obtained from American Type Culture Collection (Rockville, Md.). Cells are grown in Eagle minimal essential medium (MEM) supplemented with 10% fetal bovine serum. Freshly confluent monolayers were washed twice with MEM and then incubated with fresh medium for 24 h in the presence of actinomycin D; a known inhibitor of transcription or in the presence of gemfibrozil as an inducer of certain genes expression. Cell viability is routinely monitored by trypan blue exclusion and lactate dehydrogenase leakage. Cellular mRNA is then isolated from HepG2 or Hep3B cells and hybridized with microarray. Differential expression is analyzed and data as a comparison template is fed into a computer. A new drug with unknown function is then assayed and its is analyzed by comparison with set templates. A person skilled in the art would know how to use the protocols similar to one outlined in this example to determine the nature of an effect produced by any other drug on the levels of expression of a given gene or cluster of genes.

Drugs often have side effects that are in part due to the lack of target specificity. However, standard in vitro assay often misses the information on the specificity of a compound. In contrast, a genome reporter matrix reveals the spectrum of other genes in the genome also affected by the test compound. In considering two different compounds both of which induce the gene of interest, if one compound affects the expression of only 10 other genes and a second compound affects the expression of 100 or more other genes, the first compound is, a prioi, more likely to have fewer side effects. Because the identities of the primary genes of interest are known or determinable, information on other affected genes will be informative as to the nature of the side effect or genetic linkage to the first set of genes. A panel of genes can be used to test derivatives or analogs of the lead compound to determine which of the derivatives or analogs have greater specificity than the first drug or compound.

Alternatively, a test compound may not affect the response of a cell in an vitro assay or may not affect the expression of a gene. In the traditional drug discovery, a compound that does not display any activity will not provide any useful information. However, a compound having any significant effect on a biological process generally has some consequence on gene expression pattern. A genome reporter matrix can thus provide two different kinds of information for most compounds. In some cases, the identity of reporter genes affected by the inhibitor evidences to how the inhibitor functions. For example, a compound that induces a cAMP-dependent promoter in yeast may affect the activity of the Ras pathway. Even where the compound affects the expression of a set of genes that do not evidence the action of the compound, the matrix provides a comprehensive assessment of the action of the compound that can be stored in a database for later analyses. A library of such response profiles will be established. For example, if the database reveals that compound X alters the expression of gene Y, and a paper is published reporting that the expression of gene Y is sensitive to, for example, the inositol phosphate signaling pathway, compound X is a candidate for modulating the inositol phosphate signaling pathway. In effect the genome reporter matrix will readily provide an information on a gene in relation to a compound that may already have been found to affect the expression of that gene. This tool dramatically shortens the research and discovery phase of drug development, and effectively utilizes the value of the publicly available research databases on all genes.

(7) Determining Financial Performance of Stocks.

Investors seek the highest possible investment return with minimum risk. Heavy investments in common stock produces high returns, for example, but these returns are volatile, and losses due to stock volatility may severely impact the financial gain. It is difficult, however, to determine what mix of asset classes and in what proportion may produce the best results at an acceptable level of risk. Various methods are currently used by financial managers of stocks in an attempt to maximize return. For example, one such method of solving the problem of maximizing return involves developing the asset allocation likely to produce the highest return at a given level of performance volatility. This method, however, is not a specific solution and therefore may not produce the best results for a given investor. Another approach is to develop the asset allocation which, within a stipulated time horizon at the calculated contribution level, will lead to an acceptable probability of achieving a selected funded ratio of assets to liabilities. Despite existing financial and mathematical model the risks are still real and in some cases are not acceptable.

The instant invention provides a novel approach for managing risks of financial investments. The disclosed model as described in detail in relation to identification of genetic linkage to a disease is easily adaptable within the conditions of a financial market. By setting instant membership rules based on one or more measurements of stock performance one skilled in the art can easily identify or predict the members of latent classes.

(8) Recognizing Handwriting

One of the areas being explored for utilization of the advantages of fuzzy logic systems is in optical character recognition (hereinafter also referred to as “OCR”). In an optical character recognition application, slight variations in printing cause changes in character height and width; in addition, misfeeding the document can skew the character image, making the edges of character segments hard to detect because of overlaps in the slices. Therefore, scale, translation and rotation-invariant recognition is necessary for the optical characters. A fuzzy logic system is inherently well-suited for dealing with imprecise data and processing rules in parallel. Thus, there is also a need in the art for a technique for combining genetic algorithms with fuzzy logic systems to design a more efficient OCR application (see for example U.S. Pat. No. 5,727,130 to Hung)

Throughout this application, various publications and patents have been referenced. The disclosures in these publications or patents or references therein are incorporated herein by reference in order to more fully describe the state of the art.

Although the foregoing invention has been described in some detail by way of illustration and example for purposes of clarity of understanding, it will be readily apparent to those of ordinary skill in the art in light of the teachings of this invention that certain changes and modifications may be made thereto without deviating from the spirit or scope of the appended claims.

What is claimed is:

1. A method for classifying a plurality of objects comprising the steps of:
(a) providing one or more observations on the objects;
(b) assigning observations as a matrix in a multidimensional space, said matrix having at least two directions for each object of the interest in said space;
(c) identifying latent classes of each object according to a formula
$$f ( {\vec{Y}}_{{j}_{1} , … , {j}_{K}} ) \vert {\{ {j}_{k} ∈ {S}_{k {m}_{{j}_{k}}} \}}_{k = 1}^{K} \sim G [ h ( k , {j}_{k} , {\{ {\{ {S}_{k m} \}}_{m = 1}^{{M}_{k}} \}}_{k = 1}^{K} ) ] ;$$
(d) calculating the likelihood that each object of the interest belongs to identified latent classes.

2. The formula of claim 1 wherein kε{1, . . . , K} indexes the directions of the multidimensional space; jₖε{1, . . . , Nₖ} identifies an object in direction k; Nₖ is the number of objects in principal direction k; \overline{Y}ⱼ_{₁}_{, . . . , j}_{_{K}} is a vector of one or more observations on a set of objects {j₁, . . . , j_{K}}; mε{1, . . . , Mₖ} indexes latent classes in direction k with Mₖ being the number of latent classes in direction k; Sₖₘ is a latent class m in direction k; G[·] is a specified univariate or multivariate distribution; and f(·) and g(·) are specified functions.

3. The method of claim 1 wherein said objects are classified simultaneously or sequentially.

4. A method for identifying one or more genes linked to a cellular phenotype comprising:
(a) recording in a matrix one or more measurements on each of the genes subjected to a series of experimental or observational conditions, said measurements forming a first direction in a multidimensional space;
(b) providing measurements on a cell or tissue samples subjected to the essentially same series of experimental or observational conditions as in step (a), said measurements forming a second direction;
(c) identifying latent classes of the genes in the first direction, and latent classes of cell or tissue samples in the second direction according to formula:
log(Yᵢⱼ)iεSₘ,jεGₗ˜N[tᵢₗ+ƒ(αₘᵢβᵢⱼ,γₘₗ),σ²], and
(d) calculating the likelihood that each gene is a member of each identified latent class for the first direction, while also calculating, simultaneously or serially, the likelihood that each cell or tissue sample is a member of each identified latent class for the second direction.

5. The formula according to claim 4 wherein N[·] refers to a Gaussian distribution; Sₘ is a latent class m in the first direction; Gₗ is a latent class 1 in the second direction; and ƒ(αₘᵢ, βₗⱼ, γₘₗ) is a function of the mean parameters of a sample category, gene category, or both.

6. The method according to claim 4 wherein the cellular phenotype comprises a disease, a cellular process, a physiological pathway, a signaling pathway, a protein expression, a drug effect, or combination thereof.

7. The method according to claim 4 whereby disabilities, medications, comorbidities, laboratory results, and clinical characteristics are linked to a clinical condition in a host.

8. The method according to claim 4 whereby laboratory and observational measurements are linked to physical processes in inorganic substances.

9. The method according to claim 4 whereby chemical substances are linked to their respective pharmacological activities.

10. The method according to claim 4 whereby a financial performance of stocks is identified.

11. A method of determining in a sample a gene or cluster of genes linked to a disease using a microarray, said microarray including at least one known nucleic acid sequence, an expression and position information, comprising:
(a) extracting expression and position information to generate a set of data corresponding to at least one dimension;
(b) assigning in a computer to each dimension of the gene or cluster of genes a numerical value;
(c) generating in a computer an information algorithm for said extracted information to provide a linking pattern for said gene or cluster of genes; and
(d) determining whether the gene or cluster of genes in a sample are linked to the disease by extrapolating from the dimension-based numerical values.

12. The method according to claim II wherein the information algorithm is constructed in accordance with the formula log(Yᵢⱼ)|iεSₘ, jεGₗ˜N[tᵢₗ·ƒ(αₘₜ, βᵢⱼ, γₘₗ), σ²], wherein i and j are as the expression data by gene and sample respectively; m and l are latent classes on the corresponding dimensions; the t refer to gene expression intensity parameters; and various forms for the function ƒ are chosen.

13. A method for identifying in a library a gene or set of genes linked to metastatic properties of a cancer comprising the steps of:
(a) providing a nucleic acid material from a suspected cancerous sample;
(b) hybridizing the sample-derived probes to the library;
(d) detecting the differences between hybridization results of the sample and a reference standard;
(e) recording the differences to form a first set of data;
(f) analyzing protein expression data to form a second set of data; and
(g) combining said first set of data and said second set of data to identify the gene or set of genes which govern metastatic properties of the cancer.

14. A method for predicting a metastasizing potential of a cancer, comprising:
(a) providing a tissue sample from a subject;
(b) recording predictive parameters, wherein the predictive parameters are univariate or multivariate morphometric descriptors; and
(c) predicting the metastasizing potential of the cancer by a statistical comparison of the recorded predictive parameters with predictive parameters of a reference sample.

15. The method according to claim 13, wherein the morphometric descriptors are selected from the group comprising optical density, object size, object shape, object color, amount of DNA or RNA, angular second moment, contrast, correlation, difference moment, inverse difference moment, sum average, sum variance, sum entropy, entropy, difference variance, difference entropy, maximal correlation coefficient, coefficient of variation, peak transition probability, diagonal variance, diagonal moment, second diagonal moment, product moment, triangular symmetry, sum entropy, standard deviation, cell classification (1-Hypodiploid, 2=Diploid, 3=S-Phase, 5=Tetraploid, 6=Hyperploid), blobness, perimeter, DNA index, maximum diameter, minimum diameter, elongation, run length, configurable run length and combination thereof.

16. A method of screening for a drug that modulates an expression of a gene or cluster of genes in a cell of interest comprising the steps of:
a) exposing said cell to said drug;
b) analyzing the gene expression in said cell, and
c) comparing by the method of claim 4 the difference in gene expression of a drug-exposed cell to gene expression of a cell not exposed to the drug or exposed to a drug with known properties.

17. A method for identifying a gene or set of genes linked to a disease of interest comprising the steps of:
a) registering measured observations of the gene or set of genes as variables associated with said disease at a zero time;
b) describing the variables as a matrix in a multidimensional space, wherein each variable represents at least one first and least one second dimension in said space;
c) carrying out, simultaneously or at later times, a series of experimental observations;
d) determining projections of the experimental observations onto the first and second directions, whereby a multivariate model is obtained;
e) updating during the course of the multivariate analysis at least the first and second directions of the matrix in multidimensional space, whereby the multivariate model provides the likelihood of the gene or set of genes being linked to the disease of interest.

18. The method of claim 1 wherein said method is used for identifying genes linked to cell or tissue samples collected from a host having or suspected to have a disease comprising the steps of:
(a) assigning in a matrix one or more measurements on each of the genes over a series of experimental or observational conditions;
(b) having genes to form a first direction in a multidimensional space;
(c) allowing cell or tissue samples collected under differing experimental conditions to form a second direction in a multidimensional space;
(d) identifying latent classes of genes in the first direction and latent classes of cell or tissue samples in the principal direction;
(e) calculating the likelihood that each gene is a member of each latent class identified for the first principal direction; and
(f) calculating a likelihood that each cell or tissue sample is a member of each latent class for the second principal direction.

19. A method for classifying a plurality of objects in an image comprising the steps of:
a) inputting at least two distinct images of said objects;
b) extracting a plurality of characteristics from the images, whereby at least one characteristic of the object in one image is correlated to another characteristic of the object from another image; and
c) generating the classification result according to object membership rules, which represent a relation between the plurality of characteristics and said at least two images.

20. A method of generating membership rules for objects of interest by using a computer, said method comprising the steps of:
(a) placing measurements of a first set of objects into a database of said computer, wherein members of said first set of objects, individually, do not necessarily have any hierarchical attributes or characteristics in common;
(b) introducing measurements of a second set of objects into a second database of said computer;
(c) generating the membership rules by including members of said first set of objects and excluding those members of said second set of objects whose individual measurements match with corresponding individual measurements of objects of said first set of objects; and
(d) updating said membership rules by introducing measurements of additional sets of objects and adjusting matching criteria.

21. A method for identifying a gene or set of genes linked to metastatic properties of a tumor comprising the steps of:
(a) providing a sample from suspected tumor;
(b) extracting an experimental genetic data;
(d) detecting the differences between extracted genetic data from the sample and a reference standard; and
(e) generating mathematically an acceptance criteria which allows to link metastatic properties of the cancer to the gene or set of genes.

22. A method of identifying among a plurality of genes of known and unknown function those genes that are linked to a condition of interest, comprising:
(a) providing a mathematical model which utilizes the input data to set rejection margins;
(b) entering an experimental data from the plurality of genes of known and unknown function; and
(c) selecting genes linked to the condition of interest based on an acceptability criteria of the mathematical model.

23. A method for analyzing an image of a plurality of objects arranged as an output signal matrix by comparing to a stored output signal matrix database which sets membership rules for the objects of interest, comprising steps:
(a) constructing a stimulated physical matrix comprising an ordered array of objects having X and Y coordinates;
(b) detecting the physical signal at each said object of the physical matrix;
(c) transforming each said physical signal to generate a corresponding electrical output signal;
(d) storing each electrical output signal in an output signal matrix database associating each output signal with the X and Y coordinates of the corresponding physical matrix unit; and
(e) determining the membership of the objects of interest by comparing the output signal matrix database of step (d) with the stored output signal matrix database.
